(19) United States
(12) Patent Application Publication
(10) Pub. No.: US 2015/0157505 A1
(43) Pub. Date: Jun. 11, 2015

(54) DEVICES AND METHODS FOR GENERATION OF SUBSURFACE MICRO-DISRUPTIONS FOR OPTHALMIC SURGERY AND OPTHALMIC APPLICATIONS

(71) Applicant: Joseph Neev, Laguna Beach, CA (US)

(72) Inventor: Joseph Neev, Laguna Beach, CA (US)

(21) Appl. No.: 14/560,666

(22) Filed: Dec. 4, 2014

Related U.S. Application Data
(63) Continuation-in-part of application No. 14/016,892, filed on Sep. 3, 2013.
Application No. 14/016,892 is a continuation-in-part of application No. 12/448,987, filed on Jul. 17, 2009, now Pat. No. 8,523,926.
Application No. 12/448,987 is a continuation-in-part of application No. 13/248,001, filed on Sep. 28, 2011, now abandoned.
Application No. 13/248,001 is a continuation-in-part of application No. 13/565,398, filed on Aug. 2, 2012.
Application No. 13/565,398 is a continuation-in-part of application No. 12/239,716, filed on Sep. 26, 2008.
(60) Provisional application No. 61/387,010, filed on Sep. 28, 2010.
(60) Provisional application No. 61/048,503, filed on Apr. 28, 2008.

(51) Int. Cl. A61F 9/008

(52) U.S. Cl. CPC A61F 9/00802; A61F 9/00804; A61F 9/00814; A61F 9/00827; A61F 9/0084; A61F 2009/00872

(57) ABSTRACT

A Device and a method for using laser energy for treatment of ophthalmic tissue. The device comprises an energy source capable of generating short bursts of energy at a range of pulse repetition rates. The method comprises surface and three dimensional interactions for therapeutic use and/or to modify or remove tissue from ophthalmic targets. A device comprises an energy source capable of generating short bursts of energy at a variable pulse repetition rates. The repetition rates range from a single shot to several hundred Mega-Hertz so that selective, three dimensional interactions with a volumetric modified zone within targeted tissue of the eye issue.

### CROSS-REFERENCE TO RELATED APPLICATIONS

The present applications claims priority from and is a continuation-in-part of U.S. patent application Ser. No. 14/016,892, filed Sep. 3, 2013, which is a continuation-in-part from U.S. patent application Ser. No. 12/448,987, filed Jul. 17, 2009, which claims priority to U.S. Provisional Application Ser. No. 60/885,852, filed Jan. 19, 2007, and U.S. Provisional Application Ser. No. 60/888,258, filed Feb. 5, 2007, and U.S. Provisional Application Ser. No. 60/901,950, filed Feb. 17, 2007, and U.S. Provisional Application Ser. No. 60/904,247, filed Feb. 28, 2007, and U.S. Provisional Application Ser. No. 60/904,415, filed May 27, 2007, and U.S. Provisional Application Ser. No. 60/946,944, filed Jun. 28, 2007, and U.S. Provisional Application Ser. No. 60/978,189, filed Oct. 8, 2007; and this application is a continuation-in-part of U.S. patent application Ser. No. 13/248,001, filed Sep. 28, 2011, which claims priority to U.S. Provisional Patent Application Ser. No. 61/387,010, filed Sep. 28, 2010, all which are hereby incorporated by reference in their entireties.

And this CIP application is a continuation-in-part which claims the benefit of the U.S. patent application Ser. No. 13/565,398, filed Aug. 2, 2012, which claims priority to U.S. Provisional Patent Application Ser. No. 61/039,842 entitled “DEVICES AND METHODS FOR TISSUE PROTECTION AND DISEASE CONTROL DMTPDC”, filed on Mar. 27, 2008, and U.S. Provisional Patent Application Ser. No. 61/056,150 entitled “A DEVICE AND METHOD FOR TREATMENT OF SPINAL CORD INJURIES AND OTHER SURGERIES REQUIRING HIGH PRECISION AND ABILITY TO DIFFERENTIATE BETWEEN TISSUE TYPES”, filed May 27, 2008, and U.S. Provisional Patent Application Ser. No. 61/060,516 entitled “METHODS AND DEVICES FOR MODIFYING MATERIALS”, filed Jun. 11, 2008, and U.S. Provisional Patent Application Ser. No. 61/093,236 entitled “MULTIPLE APPLICATIONS OF ULTRASHORT PULSE TECHNOLOGY”, filed Aug. 29, 2008, and U.S. Provisional Patent Application Ser. No. 61/094,375 entitled “EYE SURGERY DEVICE AND METHOD”, filed Sep. 4, 2008, and U.S. Provisional Patent Application Ser. No. 61/121,884 entitled “DEVICES FOR TREATING TISSUE AND ENERGY”, filed on Dec. 11, 2008, and U.S. Provisional Patent Application Ser. No. 61/140,935 entitled “SUBSURFACE FLAP-FREE CORNEA MODIFICATION”, filed on Dec. 27, 2008; and this application is a continuation-in-part which claims priority to U.S. patent application Ser. No. 12/239,716, filed Sep. 26, 2008, which claims priority to U.S. Provisional Patent Application Ser. No. 61/048,503 entitled “A METHOD AND A DEVICE FOR REMOVING OR REDUCING THE PRESENCE OF HAIR WITH COOLING, PROTRUDING GUARDS, AND INTERACTING LOTIONS”, filed on Apr. 28, 2008, all of which are incorporated herein in their entirety by reference:

### BACKGROUND

This invention generally relates to a system and method for performing eye surgery. More specifically, the present invention relates to a system and method for performing intraocular feedback guided surgery using focused ultra-short pulse laser (hereinafter USPL).

The present invention also includes embodiments relating to tissue modifications and medical surgery. More particularly, the invention relates to a device and method for modifying the chemical, optical, thermal, mechanical or other physical properties of tissue in general and tissue components of an animal and human eye.

Laser has been used in ophthalmic surgery almost from the point of their invention in 1960. They have been used for coagulation of blood vessels in the retina and for removal of scattering cells the posterior side of implanted lenses in treatment of secondary cataract, among other applications.

Ultrashort plus laser, (hereunder, USPL. for short. USPL are generally lasers that are capable of emitting pulses in the range of several tens of picoseconds or less), have shown very unique ability to interact with tissue or non-organic material in a precise and highly controllable manner, sparing tissue from unwanted collateral damage to an extent that have not been seen with any other surgical system.

Over the past decade Excimer and USPL have been used to cut and reshape human cornea as a practical, accurate and more versatile instrument than the common metal blades or scalpels.

Utilizing the evolving technology of ultrashort pulse lasers, new laser tissue interaction regimes have been identified which could provide ophthalmic tissue interaction characteristics that are superior to conventional technologies and other, longer pulse laser systems. The major advantages of the ultrashort pulse laser (USPL) tissue ablation method are: 1) efficient ablation due to small input of laser energy per ablated volume of tissue and the resulting decrease of energy density needed to ablate material; 2) minimal collateral mechanical damage due to the efficient ablation and the short duration of the stress impulse; 3) minimizing collateral thermal damage due significant lowering of the energy deposition time and the fact that high energy density (energy per unit volume of target material) leads to a reduction of total energy deposition within the material required to achieve a surgical goal; 4) the ablation threshold and rate are only slightly dependent on tissue type and condition; 5) extreme precision in ablation depth is achievable because only a small amount of tissue is ablated per pulse and the number of pulses can be controlled by feedback mechanisms; 6) low acoustical (operating) noise level (as compared to the acoustical noise produced by the high or other laser systems); 7) minimized pain due to localization of energy deposition and damage; 8) ability to texture surface by controlled beam profile and rastering; 9) precise spatial control: the intensity-dependent, multiphoton process self-ensures that tissue below or laterally removed from the beam focus will not experience ablative interaction. Finally, 10) Since ultrashort pulses interact strongly with all matter regardless of specific linear absorption characteristics, efficient processing of almost all tissue types is possible.

Medical Conditions and Disorders of the Eye include (but are not limited to) the following conditions:

Blepharitis: Blepharitis is inflammation of the eyelids that can manifest in the form of the rims of the eyelids becoming inflamed—making eyes red, itchy, and sore.

Cataract: A cataract is an opacity of the (eye's) natural crystalline lens. This is a very common cause of visual impairment in older people.

Chorioretinopathy: The term chorioretinopathy refers to any disease, disorder, or medical condition of the eye that involves both the choroid layer and the retina.

Choroidal detachment: The term Choroidal Detachment refers to the seperation of the choroid from the sclera (within the eyeball) due to leakage of fluid from vessels within the choroid.

Choroiditis: The term choroiditis refers to inflammation of the choroid layer of the eye.

Conjunctivitis: The term conjunctivitis refers to inflammation of the conjunctiva of the eye. It is also sometimes known as Pink Eye—another term meaning the same as conjunctivitis.

Corneal dystrophies: Corneal dystrophies are abnormal developments affecting the cornea of the eye. There are many types of corneal dystrophy, incl. e.g. epithelial membrane dystrophy, Reis-Bücklers dystrophy, Thiel-Behnke dystrophy, Lattice dystrophies, Granular dystrophy and Fuchs' endothelial dystrophy.

Cycloplegia: Cycloplegia is the term used to refer to paralysis of the ciliary muscle of eye.

Ectopia lentis: Ectopia lentis is the term used to refer to displacement of the lens (of the eye), which may be either a complete displacement or a partial displacement (which is also known as “subluxation”).

Ectropion: Ectropion is the turning-out of the eyelid, away from the eyeball.

Entropion: Entropion is the turning-in of the eyelid, towards from the eyeball, which may lead to eyelashes rubbing against the eye causing irritation called trichiasis.

Glaucoma: Glaucoma is a condition in which loss of vision (eyesight) can result from excessively high pressure in the eyeball.

Iridocyclitis: The term iridocyclitis refers to inflammation of the iris and ciliary body of the eye.

Iridodialysis: The term iridodialysis refers to a tear in the attachment of the iris and ciliary body of the eye.

Iridoplegia: Iridoplegia is the term used to refer to paralysis of the iris of the eye.

Iris Bombe: The term Iris Bombe refers to an abnormal condition of the eye characterised by the iris bulging forwards towards the cornea.

Iritis: The term iritis refers to inflammation of the iris of the eye.

Keratitis: Keratitis is inflammation of the cornea of the eye. It is generally painful and causes the eyes to water and vision to become blurred.

Keratoconjunctivitis: Keratoconjunctivitis is combined inflammation of both the cornea and the conjunctiva of the eye.

Keratoconjunctivitis sicca is dryness of the cornea and conjunctiva due to deficiency in the production of tears.

Keratoconus: Keratoconus is an abnormality of the cornea of the eye in which the cornea has a conical shape rather than the usual curvature.

Keratoglobus: Keratoglobus (also known as megalocornea) is a congenital disorder of the eye/vision in which the whole cornea bulges forward in the shape of a continuous curve.

Maculopathy: Maculopathy is a term used to refer to any abnormality of the macula of the eye, an example is bull's eye maculopathy.

Microcornea: The term microcornea refers to an unusually small cornea (of the eye), microcornea applies to corneal diameters of less than 1 cm (10 mm).

Ophthalmia: Ophthalmia is a general term that refers to “inflammation of the eye”, and particularly the conjunctiva.

Ophthalmitis: The term opthalmitis is a general term that refers to inflammation of the eye.

Ophthalmoplegia: The term ophthalmoplegia means paralysis of the muscles of the eye. There are several forms of this condition.

Optic neuritis: Optic neuritis is inflammation of the optic nerve. Severe and/or repeated occurrences may lead to optic atrophy.

Pink Eye: Pink Eye is another word for “conjunctivitis”.

Ptosis: Ptosis (also known as blepharoptosis) is drooping of the upper eyelid, so that an upper eyelid rests at a lower position than is normal. Either one eye or both eyes may be affected.

Retinal detachment: Retinal detachment (also known as “a detached retina”) refers to the separation of the inner nervous layer of the retina of the eye from the outer pigmented layer of the retina, to which it is normally attached.

Retinal vascular occlusion: Retinal vascular occlusion is the blockage of a blood vessel connected to the retina of the eye, either blockage of an artery supplying blood to the retina (retinal artery occlusion) or blockage of a vein carrying blood from the retina (retinal vein occlusion).

Scleritis: Scleritis is inflammation of the sclera (which is also known casually as the “white of the eye” due to its colour).

Squint: The terms squint (the condition also being known as strabismus and as heterotropia) refers to abnormal alignment of the eyes.

Trichiasis: Trichiasis is a condition in which eyelashes rub against the eyeball causing discomfort and, in some cases, ulceration of the cornea.

Uveitis: Uveitis is a term that refers to inflammation of any part of the uveal tract of the eye.

Uveoparotid Fever: Uveoparotid Fever is another term for “uveoparotitis”.

Uveoparotitis: Uveoparotitis is a term that refers to inflammation of the iris, ciliary body, and choroid components of the eye but “uveoparotitis” also involves swelling of the parotid salivary gland.

### SUMMARY OF THE INVENTION

The present invention provides apparatus, systems and methods in which ultrashort pulse lasers (USPL) are used to produce patterns of micro-disruptions, while minimizing collateral damage. In some embodiments, the micro-disruptions comprise a plurality of cavities, voids, discontinuities or disruptions in the targeted eye material

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1A is a perspective view of one embodiment that produces a plurality of energy beams using a scanner.

FIG. 1B is a cross-sectional view of a schematic of the device that produces a plurality of energy beams using a scanner.

FIG. 2 is a schematic view of ultrashort pulses used to create the subsurface modifications are generated within the femtosecond laser source 201 and are directed into the patient's eye 107 using an optical scanner 202

FIG. 3 is a flow chart showing Femtosecond Ocular tissue Modification.

FIG. 4 is front view of creating microstructures pockets 410 within the targeted tissue and targeted organ.

FIG. 5A is a front view of a region of the target or ocular tissue with a pattern of volumetric modified micro zones (PVMM) 178, can be generated in the tissue of the eye or ocular organs.

FIG. 5B is a schematic of region of the target or ocular tissue with a pattern of volumetric modified micro zones (PVMM) 178, can be generated in tissue of the eye.

FIG. 6 is schematic showing the theory behind production of subsurface disruptions including temporal pulse compression, spatial and temporal photon concentration, and absorber-initiated seed electron generation.

FIG. 7 is a schematic representation of a device that produces a plurality of energy beams using a scanner or a diffractive element.

FIG. 8 is schematic representation of a device 100 for use in laser or energy-based surgery comprises: An energy source or a laser 105, a window member 100 having a substantially flat first surface 110 facing the incoming beam, and a substantially deformable second surface 120, said second surface being able to conform to the surface of the target material 130 when pressed against the target material.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

In an exemplary embodiment of the present invention, the invention contemplates a device for treating tissue, for example, tissue of the human eye, wherein said device comprises: an energy source coupled to at least one optical component and at least one controller that operates the energy source, to generate an output beam of energy, said beam of energy generates a plurality of photomodified volumes of material at or below the surface of the eye, wherein said photomodified volumes comprise a three dimensional pattern within the tissue treated tissue or human eye, and wherein said three dimensional pattern thus generated further shows no unintended modifications in the targeted tissue of the eye further than about 5 micrometer outside the boundary of said pattern of photomodified volumes.

The above device may further comprise, a controller, said controller is configured to control the output beam such that the output energy beam comprises pulsed emission, wherein each pulse has a duration of no more than about 10.sup.−6 seconds.

The above device may further comprises said controller configured to control the output beam such that the output energy beam comprises pulsed emission, wherein each pulse has a duration of no more than about 10.sup.−9 seconds.

In further elaboration of the present invention the controller is configured to control the output beam such that the output energy beam comprises pulsed emission, wherein each pulse has a duration of no more than about 50×10.sup.−12 seconds.

The above device may further comprise an optical components comprise at least one diffractive optic component capable of producing at least 10.sup.3 interaction spots in the x, y plane of the targeted tissue.

The above device may further comprise said above mentioned controller which is capable of producing a plurality of cavities that include an overlapping photomodifications or photomodifications wherein the spacing between the edges of the photomodified tissue is from about 0.01 micrometer to about 5 mm.

In the above device, said unintended modifications may comprise one or more unintended modification from a group of modifications such as: Thermal modifications, Mechanical modifications, Chemical modifications, Structural modifications. Any other modification to the structure composition or characteristic of the native, unmodified tissue.

In an exemplary embodiment the invention describe a method for treating human eye tissue. For example, a removal of crystalline lens or the shaping of the cornea by removing part of its surface or some cornea tissue under the surface and within the cornea, or for removing or modifying tissue within the sclera, retina, humor, iris or other targets within the eye.

Such a method for modifying or removing tissue should preferably be rapid, accurate, allow large or small volume modification in a operationally reasonable time (for example within the acceptable eye surgery time as known in the present day art) and provide precision and accuracy such that, for example, tissue not designated for removal or modification is not effected and are substantially spared of unwanted modifications or unwanted effects due to the procedure being performed.

In one embodiment the invention describes a device for treating tissue and the human eye. the device comprising: an energy source coupled to at least one optical component and at least one controller that operates the energy source, to generate an output beam of energy, said beam of energy generates a plurality of photo-modified volumes photodisruption or photomodified volumes at or below the surface of the eye comprising a three dimensional pattern, the three dimensional pattern thus generated further show no unintended modifications in the targeted tissue of the eye further than about 5 micrometer outside the boundary of said pattern of photodisruptions.

The energy source controller the controller can be a computer or a microprocessor or any other programmable electronics or hard-wired electronics that can provide directions and commands to control the activity of at least one of the following: the energy source, optics, mechanical components, delivery, components that enable docking to the target to be modified, diagnostics members of the device, feedback members of the device, or any other component of the device that is involved in the preparation control and monitoring of the operation of the device.

The invention further contemplates that the controller is configured to control the output beam such that the output energy beam comprises pulsed emission, wherein each pulse has duration of no more than about 10.sup.−6 seconds.

The invention further contemplates that the controller is configured to control the output beam such that the output energy beam comprises pulsed emission, wherein each pulse has a duration of no more than about 10.sup.−9 seconds.

The invention further contemplates that the controller is configured to control the output beam such that the output energy beam comprises pulsed emission, wherein each pulse has a duration of no more than about 50×10.sup.−12 seconds.

The invention further contemplates that the optical components comprise at least one diffractive optic component capable of producing at least 10.sup.3 interaction spots in the x, y plane of the targeted tissue.

The invention further contemplates that the controller produces the plurality of cavities that include an overlapping photomodifications or photomodifications wherein the spacing between the edges of the photomodified tissue is from about 0.01 micrometer to about 5 mm.

The invention further contemplates that the unintended modification comprises one or more unintended modification from a group including: Thermal modifications, Mechanical modifications, Chemical modifications, Structural modifications. Any other modification to the structure composition or characteristic of the native, unmodified tissue.

In yet another embodiment, the invention contemplates that A device for treating a region of a targeted region of the eye, the device comprising: an energy source coupled with at least one optical component capable of directing sources energy into multiple locations in the targeted region; and a controller that operates the energy source to direct said plurality of photomodified spots below the surface of the target and create a plurality of photomodified spots below the surface in a 3-dimensional pattern within the thickness of the target without any thermal damage further than 5 um below the boundary of the plurality of photomodified spots.

The invention further contemplates that a controller member of the device is configured to configure the source output energy such that each beam has a pulse duration of no more than 10.sup.−9 seconds.

The invention further contemplates that the source energy within the device produces at least 1000 photomodified spots in in a plane perpendicular to the optical axis of the eye.

The invention further contemplates that a controller member of the output of the device produces at least 1000 photomodified spots in a plane parallel to the optical axis of the eye.

The invention further contemplates that a controller member of the device directs the energy source to creates at least one photomodified target volume within the targeted tissue wherein said changes to the targeted volume result in changes to the mechanical characteristics of the targeted eye tissue.

In a further embodiment of the present invention the invention further contemplates yielding at least one photomodified volumes within the targeted eye tissue so that the modified volume change the optical characteristics of the eye.

In another embodiment of the present invention, the invention contemplates a method of modifying a region of eye, comprising: providing a laser beam generator, directing the beam from the beam generator to produce a plurality of photomodified volumes at or below the surface of the eye; wherein the photomodified volumes produce no thermal damage farther than 5 micrometers from the modified tissue, whereby the plurality of photomodified volumes create scattering centers to decrease the amount of subsequent light energy penetrating the eye.

Additional embodiment of the present invention contemplates the above method wherein the photomodified volumes or spots comprise a density of at least ten (10) photomodified volumes per cubic millimeter in the region of the ocular tissue.

By the words “volume” or “a spot” the present invention means that volume of material (i.e. a volume which is not negligible or zero but nonetheless can be very small, for example of dimensions measured in about nanometers cubes, or quite large, and measured in mm cube or even cm cube). Wherein said non-vanishing volume of material is, as a result of the laser-tissue interaction goes through an irreversible changes to it properties. For example, such irreversible changes to the material properties can include one or more of the following changes:

chemical changes of the material, physical changes of the material, changes to viscoelastic properties of the material, changes to optical properties of the material, thermal properties of the material, chemical and physical breakdown of the material, disintegration of the material, ablation of the material, melting of the material, and vaporization of the material;

Additional embodiment of the present invention contemplates the above method wherein the photomodified volumes or spots have a diameter ranging from about 0.01 micrometer to about 70 micrometer.

Additional embodiment of the present invention contemplates the above method wherein the photomodified volumes or spots have a diameter ranging from about 0.1 micrometer to about 40 micrometer.

Additional embodiment of the present invention contemplates the above method wherein the photomodified volumes or spots have a diameter ranging from about 0.1 micrometer to about 20 micrometer.

Additional embodiment of the present invention contemplates the above method wherein the photomodified volumes or spots have a diameter ranging from about 1 micrometer to about 10 micrometer.

Additional embodiment of the present invention contemplates the above method wherein the photomodified volumes or spots are at least 90% non-ablative.

Additional embodiment of the present invention contemplates the above method wherein the photomodified volumes or spots are at least 90% ablative.

Additional embodiment of the present invention contemplates the above method wherein the photomodified volumes or spots are characterized by having a spacing between the edge of adjacent photomodified volumes ranging between about 0.01 micrometer to about 10 mm.

Additional embodiment of the present invention contemplates the above method wherein the photomodified volumes or spots which are characterized by having at least some overlap between adjacent photomodified volumes or spots.

Additional embodiment of the present invention contemplates the above method wherein the photomodified volumes or spots further comprising creating the plurality of volumes or spots are below the surface of the eye and form a multilayer pattern wherein the density (number of spots per unit volume) of said laser-tissue interaction volume or spots is at least one thousand (1,000) per mm cube.

In the present invention, when the specifications or claims describe “photomodified volumes or spots” have a certain diameter, the statement implies a corresponding volume of arbitrary shape with about the same volume as a sphere with the described diameter. That is, if a claim or a portion of the specification states that a photomodified volume has a diameter of about 40 micrometer, the invention contemplate ANY photomodified volume of any shape such that the actual dimension of said photomodified tissue comprise approximately the dimension of a volume of Pi*R2, i.e. in the example of 40 um diameter which corresponds to a radius (R) of 20 micrometer, so the volume is Pi*(20 um)2. (where Pi is the well-known mathematical numerical constant approximately equal to 3.14).

Thus when the present invention speaks of photomodified volumes of about 40 um in diameter it implies ANY conceivable shape of a photomodified volumes which when measured will yield a photomodified tissue volume of approximately Pi*(20 um)2.

An object of the present invention is to provide apparatus and a method for performing therapeutic and corrective surgery on an eye using Ultrashort Pulse Lasers (hereinafter USPL) and/or Ultrashort Pulse Electromagnetic (hereinafter EM) Radiation where high precision and high accuracy are achieved using repeated position measurements that is used as a feedback signal during the procedure.

An object of the present invention is to provide apparatus and a method for performing therapeutic and corrective surgery on an eye using Ultrashort Pulse Lasers (hereinafter USPL) and/or Ultrashort Pulse Electromagnetic (hereinafter EM) Radiation where high precision and high accuracy are achieved using repeated position measurements that is used as a feedback signal during the procedure.

In one embodiment, the feedback method is based on the well-known imaging approach of optical coherent tomography (hereinafter OCT).

An OCT scan comprises an incoherent light source in transmitted into a material. The light enters the material and scattered. By performing an interferometeric measurement between the incoming light and the back scattered light, the location of the scattering event along the optical axis could be evaluated.

USPL is a laser generating radiation which is transmitted at extremely short pulses—generally such pulses are shorter than about 50 picoseconds. Once the USPL energy is concentrated into a small volume (for example, such a volume can be an approximately spherical volume of few microns in diameter) the enormous electromagnetic power causes an immediate disintegration of the material present at the focal spot due to the well-known mechanism of photodisruption, ablation, or otherwise modify the material.

If photo-disruption or ablation follow the USPL-tissue interaction, an ionized gas known as plasma is formed followed by an expansion of said gas resulting in the creation of a small cavity inside the material. If the USP source uses radiation with wavelengths that are substantially not absorbed by the targeted tissue, the resulted interaction is well confined and substantially no thermal or mechanical damage is inflicted to any of the surroundings.

USPL can be used to create a series of small spherical cavities inside the cornea of an eye in order to create an incision. This process is currently being used in order to create a corneal flap during a LASIK surgery. In order to achieve a sufficient accuracy during this procedure, the patient's eye is firmly kept in place by using a suction ring, and the cornea is flattened using a contact window. The cornel flattening process can be resulted in long term complication as the result of an increased intraocular pressure.

USP radiation can be used for many ophthalmic applications, including the performance of Lasik Surgery, cataract surgery or Crystalline Lens-related surgeries, a full refractive surgery by creating a well defined space at the stroma and reshaping the cornea as the result of removal or modification of ocular tissue, retinal surgery, optical nerve surgery, interaction with floating bodies or objects within the ocular humor, and many other therapeutic and diagnostic interactions.

However, in order to achieve such surgical goals several conditions should be achieved:

1. The surgery should be accurate and precise, modifying and removing the intended targets and avoiding damage to near-by objects or tissue of the eye that must be spared (also known as avoiding collateral damage.

(Note: Just as on the battlefield an operator of a device or a method to destroy enemy targets, always want to avoid unintended or innocent targets. Hence the term, avoiding “collateral damage”)

2. The surgeon should be able to modify, change, or remove, the desired amount of material (ranging from small to large volumes of material) in a reasonable amount of time.

3. Selectivity and guidance should be employed to avoid damaging vital tissue, hence a greater amount of accuracy needs to be achieved

To achieve goal 1, the USPL provide an excellent tool. Its pulses are the shortest commercially available, thus generating very high temporal and spatial concentration of energy within the target. Such a concentration is known as Power Density. If a very high power density can generated without large amount of total per-pulse energy, less energy is available to damage neighboring regions thus reducing the risk of collateral damage, and limiting the extent of the interaction.

Some engineers, scientists and physicians/clinician have touted the shortness of the pulse duration (e.g. tens or hundreds of femtosecond) as a big advantage of USPL in avoiding collateral damage by using what Jacques at al described as thermal energy confinement and mechanical energy confinement (i.e. the termination of the delivery of energy Before said delivered energy has time to thermal defuse or mechanical travel, e.g. via sound wave or shock wave) out of the interaction volume.

Note however, that both Nanosecond as well as Picosecond often satisfies this condition. Furthermore, even if said energy is does not emerges out of the approximate interaction zone by the time defined by the pulse duration, the deposited energy will still eventually travel out and reach an equilibrium at some time period after the interaction. (The process of diffusion of the excess energy out of the energy deliver zone is sometimes called energy transient decay).

Thus, note that pulse time duration shortness (along with the smallness of spatial extent of the beam is small) that is important to minimizing excessive damage because LESS Energy is needed to still allow us to exceed tissue interaction or tissue modification the power density threshold for modifications or interaction!

Accordingly, it is an object of the present invention to provide an improved method for performing intraocular surgery using USPL.

In some embodiments of the present invention, it is possible to avoid the use of contact window and achieving greater accuracy then the current methods.

These embodiments can be achieved by measuring repeatedly the location of the eye surface by OCT and using this measurement results as a feedback signal for the beam positioning system.

Still other embodiments of the current invention allow the use to eliminate suction ring during the surgery by constantly measuring the eye's lateral and angular location, and using these measurements as additional feedback signals for the beam positioning system.

The present invention contemplates several objectives and several embodiments. None of these embodiments should be construed as limiting the scope of the invention.

Additional embodiments of the present invention comprise methods and devices for providing fast and effective information, data, and feedback.

In one embodiment, the feedback method is based on the well known microscope imaging (hereinafter MSI).

In another embodiment, the feedback method is based on the well known imaging approach of second harmonic generation (hereinafter SHG).

In another embodiment, the feedback method is based on the well known imaging approach of third harmonic generation (hereinafter THG).

In another embodiment, the feedback method is based on the well known imaging approach of video microscopy (hereinafter VM).

In another embodiment, the feedback method is based on the well known imaging approach of luminescence spectroscopy (hereinafter LS).

In another embodiment, the feedback method is based on the well known imaging approach of Fluorescence Microscopy (hereinafter FS).

In another embodiment, the feedback method is based on the well known imaging approach of wave front analysis (hereinafter WA).

In another embodiment, the feedback method is based on the well known imaging approach of interference spectroscopy (hereinafter IS).

In further embodiment, the feedback method is based on the well known imaging approach of time-domain optical coherent tomography (hereinafter OCT).

In further embodiment, the feedback method is based on the well known imaging approach of frequency-domain optical coherent tomography (hereinafter OCT).

In further embodiment, the feedback method is based on the well known imaging approach of polarization-sensitive optical coherent tomography (hereinafter OCT).

The following description is provided, alongside all chapters of the present invention, so as to enable any person skilled in the art to make use of the invention and sets forth the best modes contemplated by the inventor of carrying out this invention.

Various modifications, however, are adapted to remain apparent to those skilled in the art, since the generic principles of the present invention have been defined specifically to provides a device and method for intraocular feedback guided surgery.

Some of the embodiments of the present invention provide a method and a system for high precision ocular feedback guided surgery. Wherein said system includes means for generating and focusing USPL radiation into a specific volume under the eye surface, and optical light reflections or scattering for measuring the relative location between the system and the patients' eye during the procedure.

The method comprises steps selected among others, from:

1. Positioning the patient such that the treated eye is kept open and facing toward the system's optical port.

2. Engaging feedback systems

3a. Engaging a mechanical suction to the eye.

3b. Steering the USPL beam focal point to its initial location under the eye surface (e.g. the cornea surface).

4. Measuring the location of the eye surface in relation to the system along the optical axis of the treatment beam (e.g. using OCT).

5. Correcting the USPL beam focal point location in response to the measurement.

6. Generating a microscopic sized material modification volumes by transmitting USPL pulse to the desired point.

7. Measuring the location of eye surface and the location of previously created cavities in relation to the system along the optical axis of the treatment beam.

8. Repeating stages 5-7 until the desired space, cut, disruption in continuity, photodisruption, or incision is created.

An OCT scan comprise an incoherent light source in transmitted into a material. The light enters the material and scattered. By performing an interferometeric measurement between the incoming light and the back scattered light, the location of the scattering event along the optical axis could be evaluated.

USPL is a laser generating radiation which is transmitted at extremely short pulses—generally such pulses are shorter than about 50 picoseconds. Once the USPL energy is concentrated into a small volume (for example, such a volume can be an approximately spherical volume of few microns in diameter) the enormous electromagnetic power causes an immediate disintegration of the material present at the focal spot due to the well known mechanism of photodisruption, ablation, or otherwise modify the material.

If photodisruption or ablation follow the USPL-tissue interaction, an ionized gas known as plasma is formed followed by an expansion of said gas resulting in the creation of a small cavity inside the material. If the USP source uses radiation with wavelengths that are substantially not absorbed by the targeted tissue, the resulted interaction is well confined and substantially no thermal or mechanical damage is inflicted to any of the surroundings.

USPL can be used to create a series of small spherical cavities inside the cornea of an eye in order to create an incision. This process is currently being used in order to create a corneal flap during a LASIK surgery. In order to achieve a sufficient accuracy during this procedure, the patient's eye is firmly kept in place by using a suction ring, and the cornea is flattened using a contact window. The cornel flattening process can be resulted in long term complication as the result of an increased intraocular pressure.

USP radiation can be used for many ophthalmic applications, including the performance of Lasik Surgery, cataract surgery or Crystalline Lens-related surgeries, a full refractive surgery by creating a well defined space at the stroma and reshaping the cornea as the result of removal or modification of ocular tissue, retinal surgery, optical nerve surgery, interaction with floating bodies or objects within the ocular humor, and many other therapeutic and diagnostic interactions.

However, in order to achieve such surgical goals several conditions must be achieved:

4. The surgery must be accurate and precise, modifying and removing the intended targets and avoiding damage to near-by objects or tissue of the eye that must be spared (also known as avoiding collateral damage.

(Note: Just as on the battle field an operator of a device or a method to destroy enemy targets, always want to avoid unintended or innocent targets. Hence the term, avoiding “collateral damage”)

5. The surgeon must be able to modify, change, or remove, the desired amount of material (ranging from small to large volumes of material) in a reasonable amount of time.

6. Selectivity and guidance must be employed to avoid damaging vital tissue, hence a greater amount of accuracy needs to be achieved

To achieve goal 1, the USPL provide an excellent tool. Its pulses are the shortest commercially available, thus generating very high temporal and spatial concentration of energy within the target. Such a concentration is known as Power Density. if a very high power density can generated without large amount of energy, less energy is available to damage neighboring regions thus reducing the risk of collateral damage, and limiting the extent of the interaction.

Some engineers, scientists and physicians/clinician have touted the shortness of the pulse duration (e.g. tens or hundreds of femtosecond) as a big advantage of USPL in avoiding collateral damage by using what Jacques at al described as thermal energy confinement and mechanical energy confinement (i.e. the termination of the delivery of energy Before said delivered energy has time to thermal defuse or mechanical travel, e.g. via sound wave or shock wave) out of the interaction volume.

Note however, that both Nanosecond as well as Picosecond often satisfies this condition. Furthermore, even if said energy is does not emerges out of the approximate interaction zone by the time defined by the pulse duration, the deposited energy will still eventually travel out and reach an equilibrium at some time period after the interaction. (The process of diffusion of the excess energy out of the energy deliver zone is sometimes called energy transient decay).

Thus it is the fact that the pulse shortness (along with the smallness of spatial extent of the beam is small) that is important to minimizing excessive damage because LESS Energy is needed to still allow us to exceed tissue interaction or tissue modification the power density threshold for modifications or interaction!

Accordingly, it is an object of the present invention to provide an improved method for performing intraocular surgery using USPL while avoiding the use of contact window and achieving greater accuracy then the current methods. These objects can be achieved by measuring repeatedly the location of the eye surface by OCT and using this measurement results as a feedback signal for the beam positioning system. Still another object of the current invention is also to avoid the use of suction ring during the surgery by constantly measuring the eye's lateral and angular location, and using these measurements as additional feedback signals for the beam positioning system.

In one embodiment the present invention provides a system for intraocular feedback guided surgery using USPL. Wherein said system includes means for generating and focusing USPL radiation into a specific volume under the eye surface, and means for measuring the relative location between the system and the patients' eye during the procedure. The present invention also provides a method for performing intraocular feedback guided surgery procedure.

To achieve Goal 2, and because, as explained above, a small amount of per pulse energy and a small interaction or modification impact is desired, a large number of pulse (some time the needed number of pulses can be millions or more) to remove or modify the desired, macroscopic, volume targeted by the surgeon. For example, if we approximate the Human Crystalline Lens as a sphere, its diameter is generally can be approximated to be on the order around 10 mm, i.e. a centimeter). To fragment, ablate, modify or emulsify even a fraction of such a relatively large organ with a per-pulse interaction volume of a diameter which is approximately only a few micrometers (a micrometer is one ten thousandth of a Centimeter) millions of pulse are needed.

If millions of pulses are delivered to a small volume, pulse to pulse residual heat is accumulated and temperature elevation takes place. Such increase in thermal build up can result in melting and damage to surrounding tissue, or other type of thermal modifications of adjacent tissue. Similarly, accumulation of other forms of energy such as metical energy or chemical energy can create unwanted mechanical or chemical modifications to adjacent tissue. For example, thermal modifications to regions near the tissue targeted for modifications or tissue regions where thermal modifications were not indented, have been demonstrated.

To avoid such undesired effects, it is important to distribute the Ultrashort pulse (USP) energy spatially and temporally so that cumulative effects of the large number of pulses used, is minimized.

Finally, to achieve goal number 3, Selectivity and guidance must be employed to avoid damaging vital tissue; hence a greater amount of accuracy needs to be achieved.

i.e. to achieve selective, correctly identify the targeted regions, guide the energy pulses to the precise and correct/intended locations, a distribute the pulses in a manner that avoids and/or minimizes unintended damage and accumulation of energy to create modifications of tissue in locations that must be protected from such modification, a smart, novel, innovative and useful device and method must be developed to allow the safe delivery and distribution, including advantageous distribution patterns in two and in three dimensions. The embodiments described in the present invention allow high pulse repetition rate that reach the level of many millions of pulse per second but distribute the pulses energy in time and space so that unwanted damage and damage not intended is reduced, and modification or harmful changes to important components of the eye do not occur.

The following description is provided, along with all embodiments of the present invention, so as to enable any person skilled in the art to make use of the invention and sets forth the best modes contemplated by the inventor of carrying out this invention. Various modifications, however, is adapted to remain apparent to those skilled in the art, since the generic principles of the present invention have been defined specifically to provides a device and method for intraocular feedback guided surgery.

The present invention provides a system for intraocular feedback guided surgery. Wherein said system includes means for generating and focusing USPL radiation into a specific volume under the eye surface, and means for measuring the relative location between the system and the patients' eye during the procedure. An embodiment of the present invention may comprises steps selected among others, from:

1. Positioning the patient such that the treated eye is kept open and facing toward the system's optical port.

2. Applying a liquid, fluid or gas to the surface of the eye.

3. Applying a mild pressure to the eye, for example a negative pressure provided by a suction interface that mechanically couples the laser delivery to the eye and thus modifying at least one of the properties of at least some tissue of the eye: Absorption of EM radiation within at least some of the eye tissue; Scattering of EM radiation within at least some of the eye tissue; Chemical properties of the at least some of the eye tissue; Mechanical properties of at least some of the eye tissue; optical properties of at least some of the eye tissue; Thermal properties of at least some of the eye tissue; A physical characteristics of at least some of the eye tissue.

4. Steering the USPL beam focal point to its initial location under the eye surface (e.g. a position on or within the cornea or a position on or within the crystalline lens).

5. Measuring the location of the eye surface in relation to the system along the optical axis of the treatment beam (for example using OCT).

6. Correcting the USPL beam focal point location according to the measurement.

7. Generating a microscopic sized cavity by transmitting USPL pulse to the desire point.

8. Measuring the location of eye surface and the location of previous created cavities in relation to the system along the optical axis of the treatment beam.

9. Repeating stages 4-7 until the desired space or incision is created.

In another embodiments of the present invention, a system for treatment of eye tissue is contemplated, wherein the system comprise, an energy source configured to generate pulses of EM energy capable of creating modification within the structure of eye tissue and in particular modifications to of the cornea tissue, sclera tissue, crystalline tissue or retina tissue.

Said modification of eye tissue includes but not limited to at least one of the following tissue modifications: photodisruption, ablation, cavitations bubble formation, and controlled physical, chemical, mechanical modification, or thermal modifications.

In a further elaboration of the above embodiment, the system comprises a three dimensional OCT imaging subsystem, said 3D OCT imaging subsystem is capable of providing information about ocular structures and providing information about tissue structure within the eye.

The system of the present embodiment may also optionally comprise at least one of the following: a visual imaging subsystem, a video imaging subsystem, a second harmonic generation subsystem, a luminance imaging sub-system, a LIB sub-system, a fluorescence spectroscopy imaging, a two photon imaging, a multiphoton imaging, a thermal imaging, an IR camera imaging, a chemical sensor, a spectrometer, or other imaging systems or sensors known in the art.

The system of the present embodiment should also comprise a scanning subsystem. Such scanning subsystem may optionally comprise a combination of at least some mirrors, lenses and other optical components, configured to move a single or multiple treatment beams so that their tissue modifications producing portion of said interaction beams is positioned and moved within the regions of the eye targeted for modifications. (for example, if a corneal flap is the desired tissue modification, said scanning system contemplated by the present embodiment, would move at least one of the interaction beam to a locations within the cornea such that a series of ablated spots or a series of photo-disruptions micro-cavities are created so that a flap within the tissue is created. Similarly if a fragmentation of the crystalline lens is desired said scanning system contemplated by the present embodiment, would move at least one of the interaction beam to a locations within the cornea such that a series of ablated spots or a series of photodisruption micro cavities are created within the crystalline lens tissue so that a fragmentation of the crystalline lens is achieved or series of cuts effectively cutting said crystalline lens into fragments is achieved.

In further elaboration of the above embodiment, the system further comprise a plurality of controllers which are operatively couple to at least the imaging system, the EM energy source, the imaging members, the OCT, and possibly other components of the eye tissue surgery system. Said controllers then can assist in performing the EM Tissue modification and/or optionally be programmed to perform said EM tissue modification automatically.

The present embodiment further comprises using the imaging and scanning sub-systems described above to obtain a map or an image of the tissue region designated for modifications, defining the spatial extent of the region in need of modifications (for example, the desired location of the corneal flap, the desired locations of incisions in the crystalline lens, spatial extent and boundaries of the crystalline lens, the locations of perforation needed in the sclera for the treatment of glaucoma, etc.)

Once the region designated for modification has been identified (either automatically, through the pre-program requirements fed into the controllers and comparing those to the digital data collected by the imaging subsystem described herein above, OR through a human input delivered by the operator of the system or ophthalmic surgeon to the processor and/or system computers which are part of the system control apparatus, and based on the imaging and analytical data viewed by the operator or surgeon, or a combination of the two ways to input said above mentioned identification of the region designated for modification) the system is given a command to begin its operation.

The operation of the system comprise allowing the output from the EME pulse source to emerge and be directed to towards a system scanning member or beam spatial and temporal manipulating assembly, so that it directs and move the beam focus (or other above modification threshold beam diameter location) so that a modification pattern is created in the tissue designated for modification. For example, a complete layer of cut tissue in the cornea, or a pattern of dissected tissue within the crystalline lens allowing lens fragmentation sufficient for safe and effective removal of said lens or any desired portion of said lens, or perforation pattern in the sclera for enhanced fluid transport across the sclera for treatment of conditions such as glaucoma.

The EMP source can be operated at a rate of between about 1 Hz and about 500 GHz and more preferably between 500 Hz and 100 GHz, or more preferably between about 1 KHz and about 50 GHz or, more preferably yet, between about 5 KHz and about 1 GHz. The EMP source may have a pulse energy between about 0.1 nJ and about 1 J, and more preferably between about 1 nJ and about 1 mJ, and more preferably between about 100 nJ and about 500 micro joule (uJ) and more preferably yet, between about 250 nJ and about 100 micro joule (uJ), and most preferably between about 500 uJ and about 50 uJ.

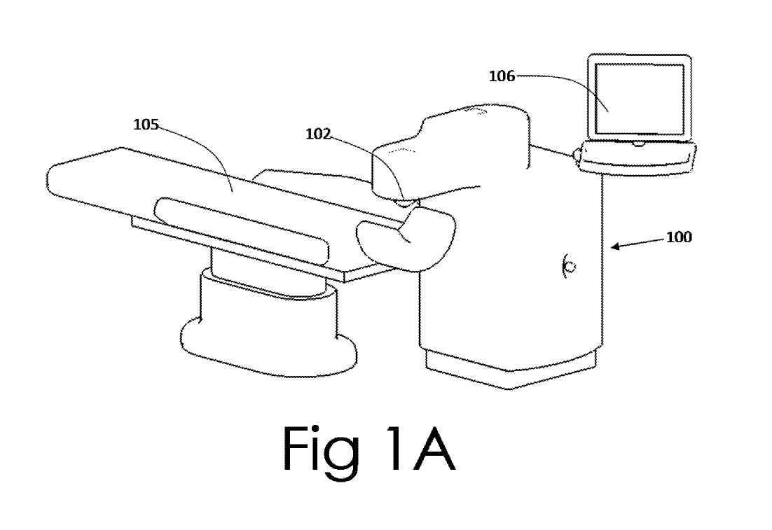

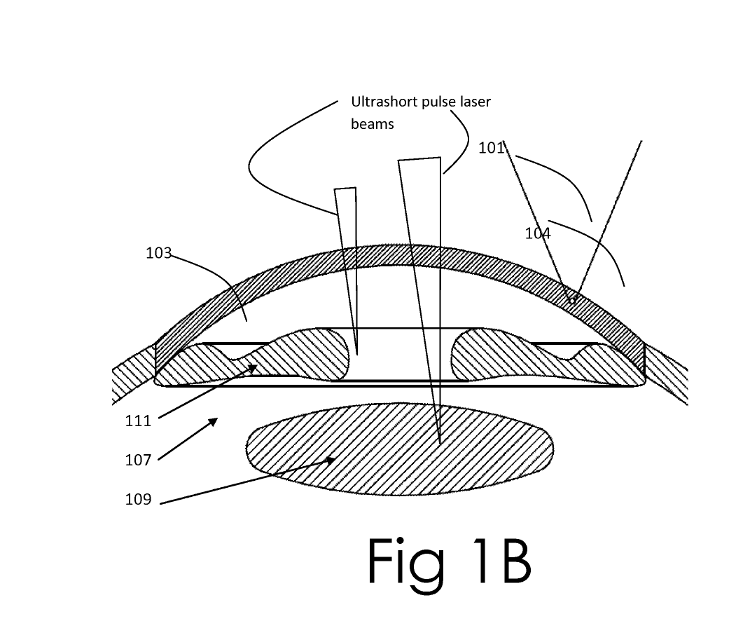

FIG. 1a is a schematic representation of a device that produces a plurality of energy beams using a scanner.

FIG. 1A and FIG. 1B shows an exemplary embodiment of the present invention. A system 100 is especially adapted for creating a subsurface modifications in the eye tissue and/or cavities inside an eye structure 103 (e.g. cornea, sclera, crystalline lens) for various ophthalmic applications (e.g. refractive surgery, cataract surgery, glaucoma surgery). The system 100 focuses ultra short pulsed laser (USPL) into the eye of a patient 107. The USPL beam 101 coming out from the optical port 102, is focused into a small focal point under the eye structure 103 surface, creating a microscopic size cavity 104 by photodisruption. By creating a series of cavities, a macroscopic cavity with a desired shape is created. In a preferred embodiment of the preset invention the patient is laying on a patient bed 105 facing toward the system's optical port 102. The system's operation is controlled by a user interface module 106.

Note that in the present invention multiple beam interaction locations is sometimes referred to as a plurality of beams or plurality of beamlets.

The definition of a light beam or a laser beam or an electromagnetic energy beam is known in the art as a directional projection of light energy (or laser energy, or EM energy) radiating from a light source (or laser energy source, or EM energy source). Thus, in the present invention the inventor adopt this definition of energy beam. Consequently. Changing the direction of the Ultrashort pulses of energy corresponds to the creation of a plurality of beams or plurality of beamlets. (Note that beams and beamlets are used interchangeably in the present invention).

Moving the laser pulses from one location to another (i.e. moving or steering the beam from one location to another) can be achieved (as was mentioned above) by using a scanner, which is often a combination of mirrors and/or lenses or other optical components (as non-limiting example concave mirrors, diffraction gratings, beam splitters, optical windows, are only a few example among many other optical and electro-optical components known in the arts). Some of these optical components, for example mirrors, can be mounted on motors, actuators, piezo electric crystals or other components known in the art as capable of moving or dispacing object in response to external signal provided by the designers and builders of the system. Thus, the present invention contemplate all known in the art methods and devices capable of moving the ultrashort pulse laser beams and the invention contemplates the possibility of using or selecting methods and devices from all known in the art possibilities.

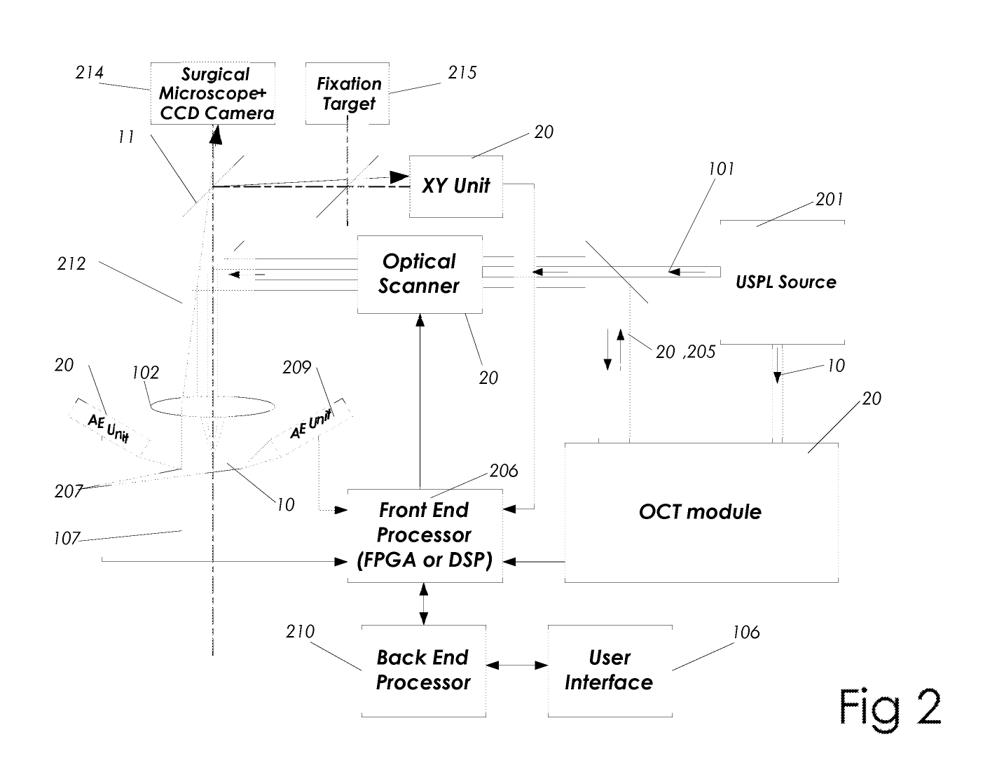

FIG. 2 illustrates another non-limiting embodiment of the preset invention wherein the location and the direction of the treated eye tissue 107 (for example, crystalline lens, cornea, retina, sclera or other targets within the eye) is measured and tracked in order to create each individual microscopic cavity 104 in its designated positions. The ultrashort pulses used to create the subsurface modifications are generated within the femtosecond laser source 201(shown in FIG. 2) and are directed into the patient's eye 107 using an optical scanner 202. In a preferred embodiment the optical scanner or another member known in the art capable of generating multiple interaction locations 202 is comprised of two rotatable mirrors for active steering of the laser beam, and movable lens for active changing of the focal point location at the targeted eye structures 103, 109, 111.

The eye measurements can be achieved using several envision embodiments. The first comprises an OCT-based apparatus for measuring the location of the targeted tissue (e.g. cornea 103, or crystalline lens 109) along the beam 101 axis. A low coherent light beam 203 coming out from an OCT module 204 and coaxially combined with the USPL beam 101 before it reaches the optical scanner. As the result that beam 203 is transmitted into the eye structure 103 along the same axis as the USPL beam 101 does. The reflected light 205 along the beam path returns the OCT module 204. By interferometeric techniques between the beam 203 and the reflection 205, the OCT module performs an A mode scan along the beam 101 axis. In one embodiment, the OCT scan is done using the FD-OCT method. In another embodiment of the present invention the USPL light source is used in addition as the low coherent light source for OCT module.

The second measurement system estimates the lateral location of the eye and the direction in which it is turned to. This is accomplished by tracking at least three microscopic sized tracking targets 207 which are located at the patient's eye. In a preferred embodiment these targets are created at the patient's eye outside the treatment zone, prior to the treatment, by the USPL beam 101. The eye's lateral movement is estimated by the XY unit 208 which measures the lateral location one of the target 207.

The eye's angular movement can be estimated using several Angular Estimator units (AE) 209. Each AE unit 209 is measuring the height of one individual tracking target 207. The differences between target's 207 heights are used for the estimation of the current eye angle.

The OCT scan results as well as the results from the XY unit 208 and the AE units 209, are processed using a front end processor 206 which accordingly controls the scanner in order to locate the USPL beam 101 focal point at its desire position in the eye's structure 103. The front end processor 206 is controlled by a back end processor 210 which receives inputs from the user interface 106 module.

A surgical microscope 214 enables the surgeon to view the treatment zone during the procedure. The microscope module is included with a CCD camera, which is connected to the back end processor 210. The CCD camera enables to employ a computer assist treatment protocols.

In order to use the same optical port 102 for the surgical microscope 214, the CCD camera 214, and the XY unit 208 together with the USPL beam 101 and the OCT beams 203 and 205 a dichroic beam splitter 212 is used. The dichroic beam splitter 212 is reflecting the USPL and OCT beams, while transmitting the light coming from the tracking target 207 and the treatment area toward the microscope, the camera 214 and the XY unit 208.

Another embodiment of the present invention is to correct hyperopic eye sight, preferably using a high repetition rate ultrashort pulse oscillator, for example an oscillator running at 88 MHz.

Another use is to modify the index of refraction of component of the eye for correction of eye sight. In this application, a laser source 200, for example a Ti: Sapphire, oscillator, running at 88 MHz with a pulse energy of for example from about 0.01 nJ to about 100 microjoule, from about 0.1 nJ to about 100 nJ is focused underneath the surface of the eye to create modifications that results in sufficient refractive index changes to correct eye sight such as myopia, astigmatism, hyperopia or In this embodiment, the pulses are focused through a transparent medium of the eye to create thermal based refractive index changes without ablating, bubbles, photodisruption or any other mechanical damage or cutting.

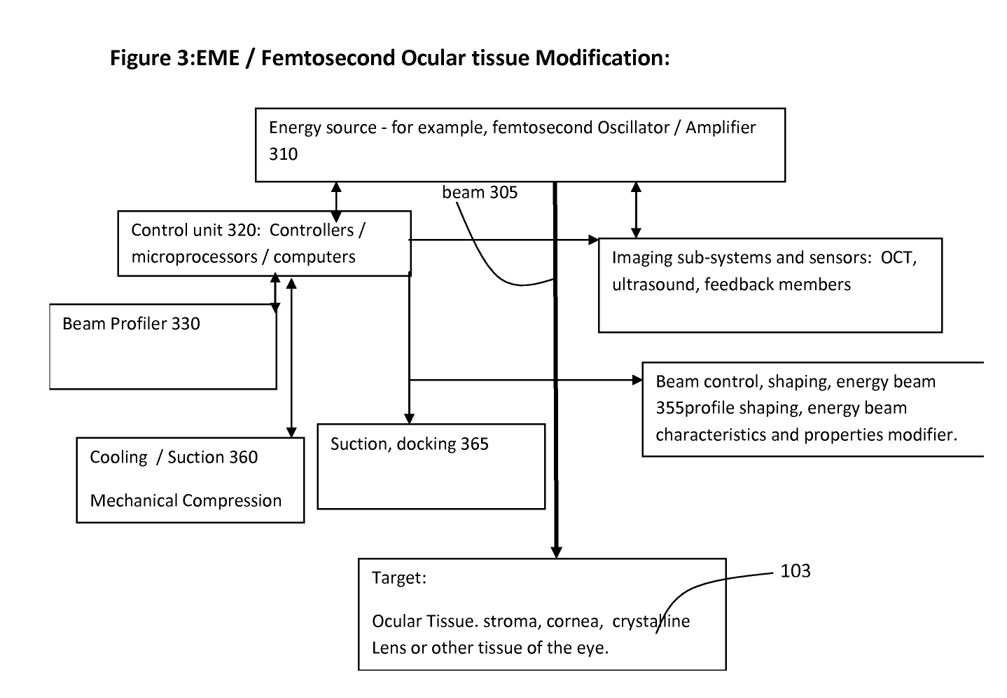

FIG. 3 shows a block diagram of the system.

FIG. 3 is a block diagram of a possible embodiment of the system contemplated by the present invention. In this embodiment an energy source, 310, for example, a femtosecond laser oscillator and optionally a femtosecond amplifier, is used as an energy source for a system for ocular tissue modification. For example, an output beam 305 emerges from the energy source 310. Said output beam is characteristics original characteristics (pulse duration, pulse repetition rate, pulse energy, etc. are determined by the energy source operation, said energy source is controlled by a control unit 320 may comprise electronic controllers, microprocessors, computers, etc.

A beam profiler 330 is used to determine the physical properties of the beam when it emerges from the source 310 and the beam controller and shaper 355 modify its properties (Spot size at the targeted region, Wavelength, pulse duration, energy per pulse, pulse rep rate, time delays, time of travel, energy profile spatial and temporal shaping, etc.).

A cooling and/or suction can be provided to the targeted material form a cooling and suction modules 360.

A docking and suction 365 can be applied to the targeted module by a docking module 365, where the docking can be a disposable lens, disposable window, patient interface, etc. and can be made from plastic or glass or other biocompatible materials known in the art.

An imaging and sensing module 375 is provided to image and monitor the target position, shape, modification of the target, progress of modifications, and for determining target characteristics such as shape, structure, composition, optical, thermal, dielectric, electric, mechanical and other properties.

Pulse Compression and Eye Surgery.

When short pulses of electromagnetic (EM) energy (or EME, for Electromagnetic Energy, of EMR, for Electromagnetic Radiation). are used, the pulse duration (pulse duration shall be designated with the Greek letter Tau, τ, can be configured to become dynamic, and change as the EM Pulse of energy approach the targeted volume.

The ability start the tissue penetration (for example a EME pulse crosses the eye surface, or cornea surface, and propagate into the eye tissue) with longer pulse duration at the targeted organ surface (for example surface of the eye) and compress the pulses as they propagate and penetrate deeper into the tissue, allow the user or the surgeon to interact with deeper tissue layers.

The advantage of penetrating the targeted organ (or any other material that allow at least some of the EM energy to propagate through it) is that longer EME pulse propagation through transparent or partially transmitting medium avoid premature concentration of power density (power density is energy per unit volume per unit time) and the occurrence of premature ablation, premature heating, or other premature EME pulse-material interaction, or EME pulse-tissue interactions. For example, white light generation, self-focusing, thermal lensing, or other nonlinear or EME intensity dependent effects.

The above mentioned effects can be avoided because less spatial focusing, or even substantially no spatial focusing, are used in delivering the energy pulses to the targeted region vicinity, and the operator or user, or the device, uses the Temporal focusing or pulse compression to bring the power energy density at about the region of the targeted volume to a power density level that is substantially above volumetric power density threshold for material or tissue modification. (Again, said modification can be thermal, ablative, photo-disruptive, evaporative, explosive, acoustic, mechanical, chemical or other forms of tissue or material modification).

Note: whenever tissue as a target material is discussed, it is to be construed as ANY type of physical material to be modified. Such physical material may or may not be tissue and may or may not be organic. Similarly, whenever the inventor discusses material as a target material it is to be construed as ANY type of physical material and/or tissue to be modified. Such material may or may not be tissue and may or may not be organic.

The present invention contemplates a device that allows interaction of EME pulses with variable spots sizes at the region targeted for modifications.

Because of the ability of the device and method of the present invention to change the pulse duration at region targeted for modification, the restrictions on spot sizes can be relaxed and a larger variety of EME spot sizes can be used.

Note: Spot size can be defined in various ways. For example spot size can be the diameter of the EME transverse extend (transverse means the direction perpendicular to the axis of EME beam propagation) wherein said extent of the EME profile drops to 0.1 of its peak value, or the distance where the EME profile drops to 1/e of its maximum value at the center of the beam, or the extent of the full width at half max (FWHM) which is well known definition used by those skilled in the art. In the present invention I will use refer to the FWHM unless otherwise specified.

Note also that in general wherever a pulse of EME is spatially converging, the volumetric power density (VPD) (i.e. EME per unit volume per unit time) is increasing as the pulse propagates through space. Wherever the VPD reaches a threshold above which tissue or material modification is initiated (for example due to concentrating enough EME photon into a small enough space and short enough time so that, for example, electrons are torn off their bound state to a nucleus or a chemical molecular bond is broken) then tissue or material modification will be initiated.

Thus, 1. Maturely modification does not have to happen at the beam focal point, it can happen before the pulse reaches the focal point if the VPD is high enough at any point BEFORE the EME pulse reaches the focal point. And, 2. Material modification is NOT confined to the beam FWHM lateral spatial extend. This is so because the ionized electrons, phonons, mechanical or thermal energy transients, or atomic and molecular byproduct form broken bonds, plasma, gases and tissue or material debris will expand the modification of the tissue or material in the region targeted to modification into a zone of modified tissue (or modified material, if the material is inorganic or other material than tissue) so that a region larger than the region of defined by the diameter of the FWHM, is modified.

Additionally, note that such a region of modified material (ablated, photo disrupted, thermally modified, chemically modified, mechanically modified, or otherwise modified region) is NOT part of the expected, intended and desired modification. This said modification zone is often known, expected, and designed for by the engineers and users who design, build and use the system or use the method, by selecting the EME pulse parameters (for example, expected FWHM at the desired region, the pulse duration at the targeted region, the EME pulse energy at the targeted region, etc.), and by known the targeted material properties(for example, the targeted material index of refraction, tensile strength, thermal properties, mechanical properties, thermal properties, optical properties, electrical properties, etc.)

Note that this said extend of the modified material is NOT unwanted damage, unintended damage or collateral damage, It is part of the interaction and a material modification desired, expected and designed for.

Collateral damage, unintended damage, undesired damage, or any kind of damage for that matter, is a the kind of modification NOT wanted by the designer, builder, and users of the system or method, an effect to be avoided because it can cause changes that will render the targeted material less valuable or create an injury, complication, morbidity, risk, danger, loss of vision or eye sight, pain, reduced visibility, inconvenience, or other long terms disability, injuries or even death. Such adverse and unwanted effects are often referred to in medicine (as well as in military context) as collateral damage (for example, damage to the optical nerve or retina when trying to create a flap in the cornea or while cutting a section of the crystalline lens. Or, in a dental surgery case, damaging the viability of the live tissue and processes in the root of a tooth, for example, by generating too much heat while removing dentin tissue around a cavity.

Some embodiments of the present invention incorporate EME pulse temporal variation (most commonly pulse duration compression, but when needed, also pulse duration lengthening) in order to increase or decrease the period of the pulse as the EME pulses propagate towards the targeted region.

Because, some embodiments of the present invention incorporate EME pulse compression (that is, designing a system or a method that cause pulses to change their time duration—this is also known as the period of the pulse), higher volumetric power density (VPD) can be achieved by compressing or shortening the duration of the pulse, and consequently, the designer of the embodiment contemplated in the present invention device and method (or the users) can employ a variety of beam spot size at the regions indented for modifications yet still achieve the modification threshold VPD with larger beam spot size if they compensate by using shorter pulse duration at the targeted modification regions.

Thus, an additional advantage is the ability of the method and device to employee a beam of a variety of spot sizes. For example, exemplary Parameters for the Beam Spot size: Wherein the beam spot size is Larger than about 1 cm but smaller than about 10 cm; Larger than about 5 mm but smaller than about 10 mm; Larger than about 1 mm but smaller than about 5 mm; Larger than about 0.5 mm but smaller than about 1 mm; More preferably yet, larger than about 0.2 mm but smaller than about 0.5 mm; More preferably, Larger than about 0.1 mm but smaller than about 0.2 mm; Preferably, Larger than about 50 micrometer but smaller than about 100 micrometer; Larger than about 25 micrometer but smaller than about 50 micrometer; Larger than about 10 micrometer but smaller than about 25 micrometer; Larger than about 5 micrometer but smaller than about 10 micrometer; Larger than about 1 micrometer but smaller than about 5 micrometer; Larger than about 0.5 micrometer but smaller than about 1 micrometer; Larger than about 0.2 micrometer but smaller than about 0.5 micrometer; Larger than about 0.1 micrometer but smaller than about 0.2 micrometer; Larger than about 50 nm but smaller than about 100 nm; Larger than about 25 nm smaller than about 50 nm; Larger than about 10 nm smaller than about 25 nm; Larger than about 5 nm smaller than about 10 nm; Larger than about 1 nm smaller than about 5 nm.

Most often, embodiments involving tissue of the human eye will deploy tissue-interaction spot sizes in the region targeted for modification of from about 50 nm to about 40 micrometer, and more preferably from about 150 nm to about 20 micrometer, and most preferably from about 0.5 um to about 10 micrometer.

A broad beam may also be used with temporal pulse compression, wherein said broad beam with said pulse temporal compression allows deeper penetration, said deeper penetration is substantially more free of non-linear effects and substantially more free of self-focusing and white light generation. The improved working parameters with the above mentioned broader or wider beam allows improved work within: In the eye; In cornea treatment such as LASIK and removal of lens in treatment of cataract.

In the environment of the eye, it is often needed to treat targets that are deep within the eye ball, in the cornea, in the lens, in the sclera, in the retina, floating bodies in the liquid humor, or other targets. In such cases, high power density pulses (for example, for femtosecond or picosecond lasers) can create nonlinear effect in the eye as they travel towards the targeted area. For example, white light generation, thermal lensing, self-focusing or other nonlinear effects. The compression methods, devices, and methods described above allow the user or doctor, ophthalmologist, to avoid such treatment problems and reach targets deep inside the targeted regions of the eye ball or other targeted materials.

Principle of operation: Correcting vision in the eye and treatment of eye ailments:

Another embodiment comprises a system and method to modify vision is contemplated. It has been described in the past by the present inventor as well as other prior art, it is known to cut flaps or subsurface lines within the cornea. The embodiments disclosed herein contemplate creating subsurface structures as described by the parameters tables shown in table 1a to table 1e, in the cornea or lens, or both in the cornea and Lens. Optionally, or additionally such structures can also be cut in sclera or other structures of the eye. The embodiments disclosed herein contemplate using such structures to modify the elastic properties or the optical properties or at least one of a group of properties of the eye using such structures 520. Among the group of such properties of the eyes are: Elastic properties, Optical properties, Refractive properties, Thermal properties, Hardness, Opacity, Absorption, Scattering, Electrical properties, other properties.

Principle of operation: Correcting vision in the eye and treatment of eye ailments:

In this embodiment the system and method to modify vision is contemplated. It has been described in the past by the present inventor as well as other prior art, it is known to cut flaps or subsurface lines within the cornea. The embodiments disclosed herein contemplate creating subsurface structures as described by the parameters tables shown in table 1a to table 1e, in the cornea or lens, or both in the cornea and Lens. Optionally, or additionally such structures can also be cut in sclera or other structures of the eye. The embodiments disclosed herein contemplate using such structures to modify the elastic properties or the optical properties or at least one of a group of properties of the eye using such structures 520. Among the group of such properties of the eyes are: Elastic properties, Optical properties, Refractive properties, Thermal properties, Hardness, Opacity, Absorption, Scattering, Electrical properties, Other properties.

Additionally or optionally, a fluid or liquid is injected to the structures thus created within the Lens or the cornea, or other structures within the eyes. Such fluid or liquid may be injected or otherwise inserted into the eye and its volume, pressure, or density, or other relevant characteristic may be adjusted to allow control (possibly even dynamic, real time, adjustable control) of the curvature of the cornea or lens or other components of the eye, to allow treatment of refractive power of the eye, focusing power of the eye, and/or corrections of such ophthalmic conditions as myopia, presbyopia, astigmatism, cataract, or other ophthalmic conditions.

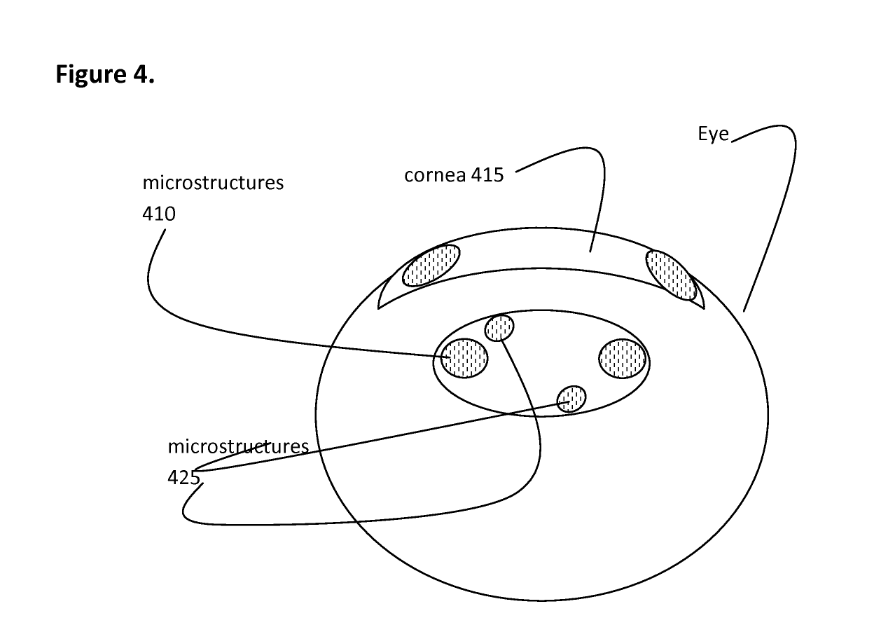

Additional embodiment for Ophthalmic applications in—(Drawing FIG. 4):

Additionally or optionally, a fluid or liquid is injected to the structures thus created within the Lens or the cornea, or other structures within the eyes. Such fluid or liquid may be injected or otherwise inserted into the eye and its volume, pressure, or density, or other relevant characteristic may be adjusted to allow control (possibly even dynamic, real time, adjustable control) of the curvature of the cornea or lens or other components of the eye, to allow treatment of refractive power of the eye, focusing power of the eye, and/or corrections of such ophthalmic conditions as myopia, presbyopia, astigmatism, cataract, or other ophthalmic conditions.

In another embodiment the energy source, for example a femtosecond laser, creates the storage for a fluid or a liquid. The Fluid or liquid can, for example, be a memory retaining polymer. A Fluid containing absorbers for enhanced absorption of the incoming energy, or fluid which is doped with absorbers, for example nanoparticles that can expand upon the delivery of a willfully triggered external signal, for example a laser signal, laser energy, light energy, acoustic energy, mechanical energy, thermal energy, or other forms of external energy that can travel through the human tissue, reach them, and cause them to expand. Depending on the position of said fluid pockets, they can either inflate or deflate the lens. For example the crystalline lens of the eye, thus causing increase or decrease in the focusing power of said lens. For example, an activation of the absorbers in pocket 620 can cause a lens to inflate and focus more. Thus the method and a device allow us to overcome and correct presbyopia. This is illustrated further with the help of FIG. 4.

One can create microstructures pockets 410 within the targeted tissue and targeted organ, in accordance with the cavities or voids dimensions specified by the embodiments disclosed herein. The embodiments disclosed herein contemplates inserting fluids, for example, doped with nanoparticles that respond to external energy and expends and perverse their shape or cool off and retract or expend in one part of the cornea 415 to stretch, and expend in another part to contract. While inactivation of the effect, can be achieved, for example, by activation of the absorbers in one part of the crystalline lens or lens (or other tissue in other ocular targets), thus causing deflation of the lens or cornea, allowing flattening of the lens (“flattening” means making the lens flatter in appearance or in curvature, i.e. more oval and flat instead of the lens being more round and more curved). Lowering of the lenses focusing power, and, for example, treatment of myopia. Thus, in effect, the one trigger 410 causes expansion and bulging, while a second trigger(s) position in a different location of the tissue or organ targeted for volumetric or curvature modification (e.g. crystalline lens or cornea). Additionally or alternatively, when an opposite effect of flattening of the organ or tissue is desired, microstructures pockets 425 positioned in other locations are activated. Hence, microstructure pockets 410 are activated to cause bulging, and microstrucutre 425 cause the opposite effect and changing the organ or tissue structure in a reverse manner (or “turning off”) of the first trigger. The two triggers work like two sets of switches with on and off switch wherein the energy is provided externally by the external energy source (e.g. laser light beam etc.)

In further elaboration of the present embodiments and principles of operation thus comprises (as shown in FIG. 4): 1) The microstructure created by the beam from the energy source can be used to generate a storage space 410 (along other storage volumes in different locations and with desired structures). Such storage space can be created with the aid of an external energy source, for example, an external fs laser or USPL and its ability to create subsurface structures in the eye (or in other tissue or body parts, or other materials and substances). 2) The insertion (for example, injection) of a fluid or liquid capable of modifying at least one property of the eye (or other tissue or body part). For example, the injection of substance that can be willfully triggered by a signal from an operator they expand. For example, a biocompatible fluid or liquid or other substance that can expand upon heating, wherein such biocompatible substance also contains a substance that can absorb a radiation from an external laser (for example, a biocompatible substance containing nanoparticles that converts said external energy into heat), and thus expending and causing the tissue (for example a lens so or a cornea) to change it shape. 3) Activation of said inserted substance by an external source, and obtaining a desired shape (and possibly function) of the treated organ. For example, insertion of nanoparticles doped polymers into pockets or voids prepared by an external energy source such as a laser, or ultrashort pulse laser, can allow the user or operator to change the shape of a lens or cornea to improve vision. 4) Such changes to tissue or organs (for example, the eye crystalline lens) can be reversible and/or adjustable as the external source can be willfully used as described herein above to modify or adjust the changes.

### Embodiment—Cavity Diagnostics

A micro-cavity biosensor monitors optical resonances in micro- and nanostructures for label-free detection of molecules and their interactions. Recent applications allow optical microcavities for nanoparticle detection, trapping and manipulation, and I will highlight different modalities for ultra-sensitive label-free bio-sensing. The embodiments disclosed herein contemplate the creation of microcavities (for example—with fs lasers) within a biological tissue, for example within the environment of the eye. Changes in light trapped within the eye are then monitored to detect the presence of atoms, molecules, proteins, and viruses within the tested environment, including insulin level.

### EXAMPLE 1

### Multi-Photon (MP) Method and Device for Enhanced Penetration Across a Barrier

A series of cavities can be generated in the z-axis direction. These can be inter-connected to create an effective channel for the delivery of drugs or medicine or for example, for the storing of drugs, medicine, nutrients and vitamins, within the tissue for controlling medical conditions such as glaucoma or floaters, for disabling muscles that control undesired eye motion or distortions of the ocular lens and transmission of light, or other ocular muscles.

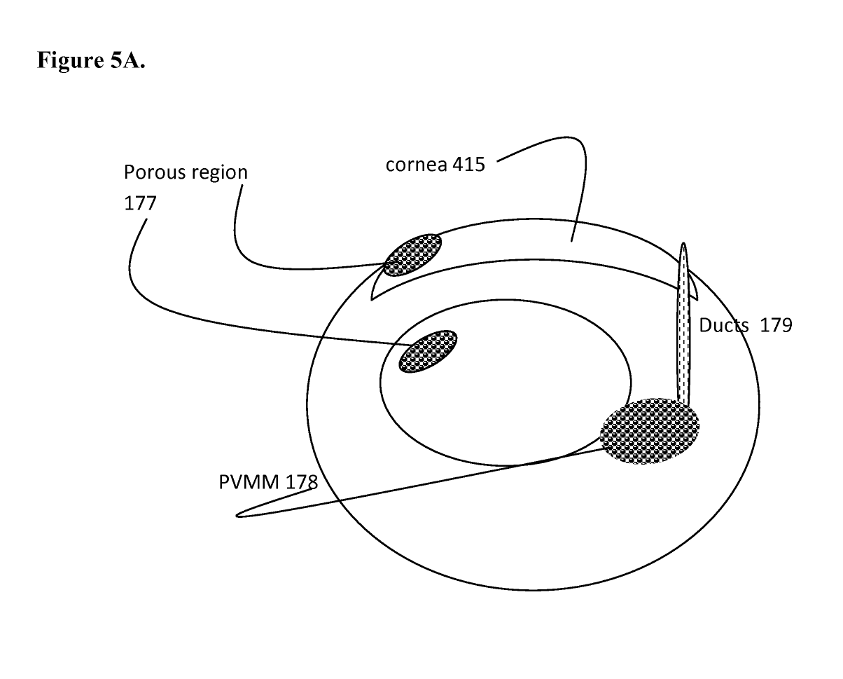

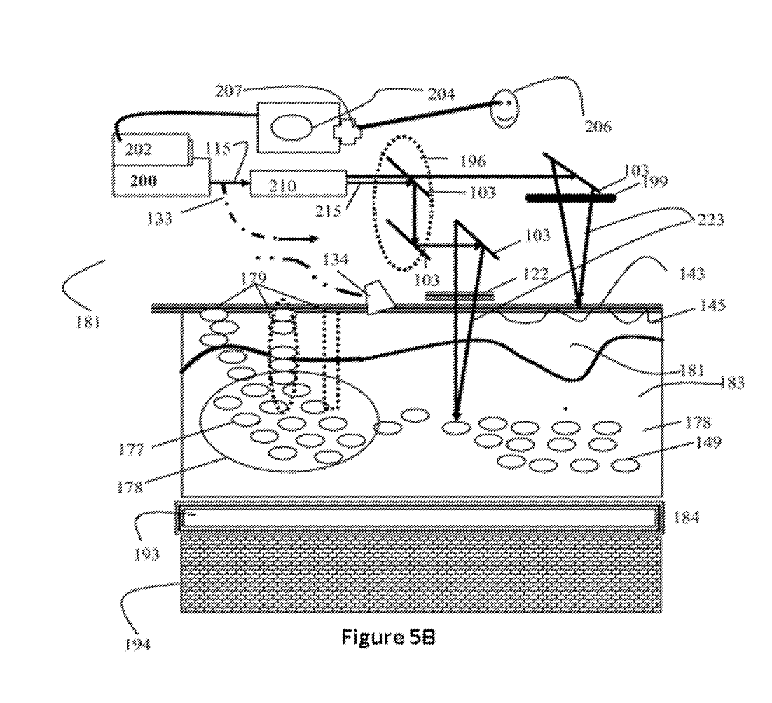

In FIG. 5a, for example, the cavities can be connected to each other or can be connected in part (some are connected and some are not) or can be an assembly of individual cavities that are not connected. The assembly of cavities thus forms a region of tissue or components of the eye with enhanced porosity. The porous region of the tissue or ocular tissue 177, can then be connected to the surface with a series of ducts or ablated holes 179 generated by the device. Ducts or pores can be generated by the device ablating from the surface down through the various layers until it arrive at the device-generated porous region 178.

As FIG. 5a also shows, such region of the target or ocular tissue with a pattern of volumetric modified micro zones (PVMM) 178, can be generated in the eye tissue layer or ocular tissue. For example, using techniques set forth herein, a PVMM 178 can be generated in the ocular lens, cornea, retina, or sclera, muscles of the ocular systems or other tissue and targeted materials.

In another preferred embodiment the ducts, conduits, and channels, 179, can be generated by filaments, transient mechanical effects such as a bubble, or self-focusing filaments. These effects can be used to extent enhance or replace some or most of the cavity generated by direct device generated cavities 177. They can be used in combination with the direct-device generated bubbles such as ultrashort pulse generated cavities to create a network of channels and ducts leading to the porous generated area or for creating pours region all by themselves.

Such porosity-generating patterns can be made from the bottom up meaning that the pattern can be generated at the bottom of the targeted tissue (for example in the anterior region of the lens or anterior region of the cornea, and then, a another pattern can be generated in the upper layers of the more posterior layers of the target, for example the layers of the cornea closer to the external surface of the cornea.

### EXAMPLE

### Multi-Photon (MP) Method and Device for Enhanced Penetration Across a Barrier

A series of cavities can be generated in the z-axis direction. These can be inter-connected to create an effective channel for the delivery of drugs or medicine or for example, for the storing of drugs, medicine, nutrient and vitamins within the tissue (for example the cornea) for controlling ailment and diseases (for example, glaucoma), for enhancing the drainage of excess fluid in the eye or regulating fluid pressure within the eye, for disabling muscles, for eliminating or removing unwanted growth such as a benign or malignant tumors, for the control of diseases such as diabetes or other eye diseases.

As an example, FIG. 5A, also shows modified regions generated by the creation of EME pulses can be connected to each other or can be connected, at least in part (i.e. where some modified regions are connected and some are not) or can be an assembly of individual cavities that may or may not be connected at the time of their creation. The assembly of interconnected cavities thus forms a region of the tissue with enhanced porosity. The porous region of the tissue or the tissue 177, can then be connected to the surface with a series of ducts or ablated holes 179 generated by the device. The ducts or pores can be generated by the device ablating or removing material from the surface downward through the various regions until it arrives at the device-generated porous region 178. Alternatively, a series of ducts, pores, or channels can be created by ablating or removing material from the device-generated pattern upward.

As FIG. 5B, also shows, such region of the target or ocular tissue with a pattern of volumetric modified micro zones (PVMM) 178, can be generated in tissue of the eye.

A preferred embodiment the ducts are generated by extending some cavities 179 upward from the porous region 178 towards the surface. The extended cavities 179 thus form a duct 179 which or extended pore which connect the surface to the porous region 178. The ducts are generated by focusing the beam below the surface of the tissue and moving the focus up until the cavities thus generated reach the surface. By using this preferred embodiment, the surface can be minimally disrupted and pores and ducts generated under the surface with minimal cutting or ablating of the surface and sometimes without even perforating or breaching the surface.

In another preferred embodiment the ducts, conduits, and channels, 179, can be generated by filaments, transient mechanical effects such as a bubble, or self-focusing filaments. These effects can be used to extent enhance or replace some or most of the cavity generated by direct device generated cavities 177. They can be used in combination with the direct-device generated bubbles such as ultrashort pulse generated cavities to create a network of channels and ducts leading to the porous generated area or for creating pours region all by themselves.

Such porosity-generating patterns can be made from the bottom up meaning that the pattern can be generated at the bottom of the region of the organ to be treated (for example in the layers in the bottom or posterior part of the crystalline lens or cornea or region of the sclera, or middle section of targeted region of the organ for example around the center of the eye lens or the middle layers of the retina) and then in a follow up action of the EME pulses, a pattern can be generated in the upper layers of the organs.

### EXAMPLE 2

### Ocular Tissue Al Filler

It is known in the art to apply eye drops and medications to the surface of the eye or to inject various beneficial compounds or medications to the eye.

According to teachings herein, porosity of an ocular tissue can be significantly increased, to create a “sponge effect” for ocular fillers, drugs, medications, or any other substance that a physician may want to store, inject, or incorporate into the ocular environment or to store in the ocular environment for later use. It is still further contemplated for users to create ducts and minutes ducts or capillaries to couple a substance from the surface of the eye or a targeted tissue to the porous regions created below the surface of the eye.

Method And Device for Treating Tissue with Very High Pulse Rep Rates.

In yet another embodiment, a low power ultrashort pulse laser or similar energy source is use to modify the index of refraction of component of the eye for correction of eye sight and ocular tissue. In this application, as a non-limiting example, a Ti: Sapphire, oscillator, running at 88 MHz with a pulse energy of for example from about 0.01 nJ to about 100 microjoule, from about 0.1 nJ to about 100 nJ is focused underneath the surface of the eye surface to create modifications that results in sufficient refractive index changes to correct eye sight such as myopia, astigmatism or hyperopia. In this embodiment, the pulses are focused through a transparent medium of the eye to create thermal based refractive index changes without ablating, bubbles, photodisruption or any other mechanical damage or cutting.

A preferred embodiment envisions direct modification to tissue can be made by creating the following pattern in the cornea or lens of the eye:

If for example a series of tissue modified spots are created, the size of the spots can then vary in diameter from about 0.01 μm to about 200 μm, from about 0.1 μm to about 0.1 mm and more preferable from about 0.2 μm to about 50 μm and more preferably yet from about 0.7 μm to about 20 μm and more preferably from about 0.8 μm to about 10 μm, and more preferably yet from about 0.8 μm to about 5 μm, 1 μm to about 5 μm, and most preferably from about 1 μm to about 2.5 μm. The pattern is made optimize the effect desired. For example, for modify the index of refraction of the eye high repetition low pulse energy ultrashort pulses are used in an energy range of 0.1 to 100 nJ, from 1 nJ to 10 nJ and pulse repetition rate of from 1 KHz to 1 GHz and more preferably from 100 KHz to 100 MHz and most preferably from 10 MHz to 90 MHz.

### EXAMPLE 4

### Pulse Compression

In yet another preferred embodiment, the embodiments disclosed herein contemplates sending an ultrashort pulse down the eye tissue layers or ocular tissue or other tissue or material. The method and device preferably aim at achieving selective interaction below the surface of a target material or tissue by using pulse compression in a medium. For ultrashort pulse, the pulse will experience compression in time due to dispersion effects as it propagates in the media. The compression of the light in time allows it to reach an above-threshold power density level at a certain distance into the tissue. A distant that can be calculated and targeted to achieve a depth of tissue modification. The pulse is compressed by the medium as it propagates down the tissue so that when it arrives at the targeted region (for example the vicinity of the hair bulb or papilla) its power density is enough to damage said target.

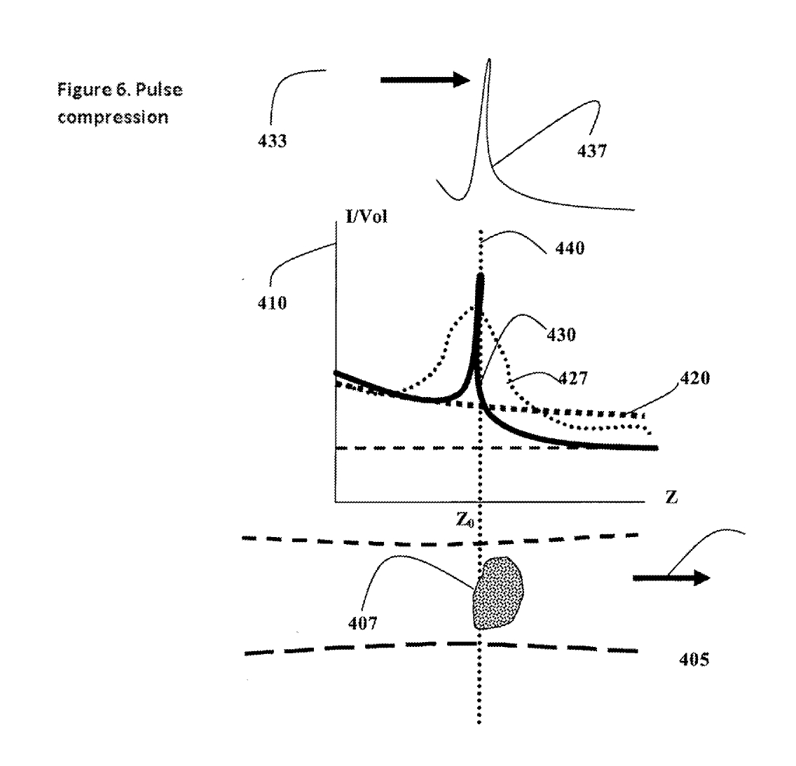

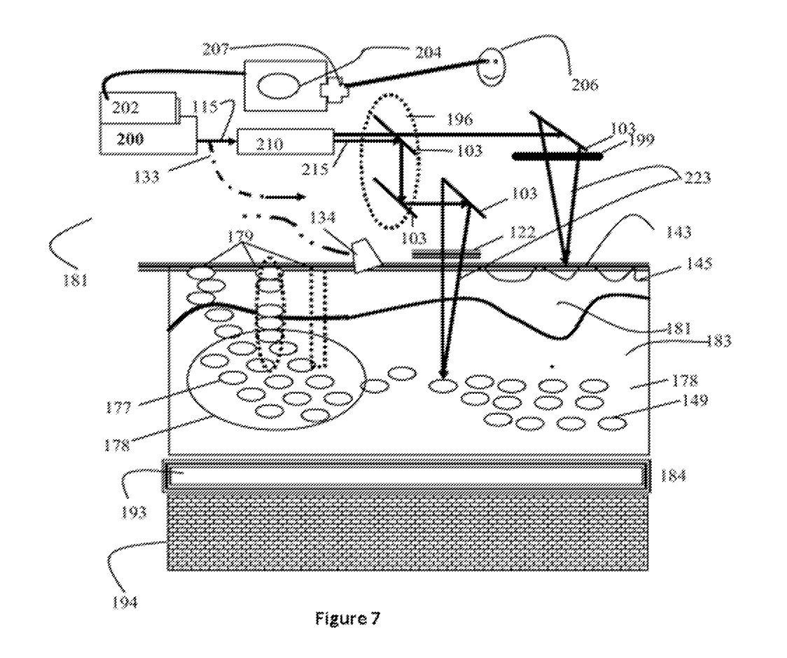

Pulse compression is illustrated in FIG. 6. The initial pulse at the surface is of shape 433, representing the uncompressed pulse. As the pulse penetrates and propagate through the tissue, it can designed to reach a compressed stage shown in 437 at depth Z0 allowing the power density (I/Volume) to reach above interaction threshold level so that the target or tissue are modified at depth Z0. As an example, a laser pulse can be directed towards the sebaceous gland region and be shaped in time so its power density is below threshold for interaction because the pulse duration is relatively long. As it propagate down the ocular tissue the pulse is compressed as described above and pulse reach above interaction power density because the pulse at depth Z0 is compressed. For example the focusing element (for example a lens) 122 in FIG. 7 can be moved or oscillate to allow the location of compressed pulse to vary in depth around the targeted region to achieve the desired tissue or healing effect (for example, photodisruption, ablation or non-ablative damage to the sebaceous gland).

High pulse repetition rate (PRR) laser energy can be applied either at the surface of the ocular tissue or below the surface of the ocular tissue to create reflective effects that will result in changes to ocular tissue coloring. Alternatively, and in a preferred embodiment, pattern induced by the high PRR pulses of electromagnetic energy either at the surface or below the surface of the ocular tissue can be used to reflect unwanted radiation or wavelength and prevent penetration into the ocular tissue or tissue or other material one wishes to protect. Thus, in a preferred embodiment, a high pulse repetition rate lasers running at a pulse rep rate of from about 10 Hz to about 100 MHz, from about 1 KHz to about 100 MHz can be used, with sufficient power density, (for example with pulse duration from about 1 microsecond to about 1 fs, from about 10 ns to about 3 fs) and pulse energy from about 0.01 nJ to about 10 J, from about 0.1 nJ to about 1 J and spot size at the surface of from about 0.01 μm to about 100 mm, from about 10 μm to about 2 mm, along with a scanner or other elements that are capable of moving the beam along the target surface of bulk volume (for example, galvo scanners, spinning polygon, or vibrating lens, among other methods known in the art for scanning or stirring beams), so that the a pattern is created that rejects or reflect certain colors or wavelengths. For example, a destructive interference in the forward direction can be created to reject wavelength in the range of from about 170 nm to about 400 nm thus reducing the penetration of cancer-causing radiation in the UV. Alternatively a reflection of sunlight or room light in the wavelength that appears to the eye in the color of tanned ocular tissue can be useful aesthetically to enhance the perception of beauty and healthy-looking ocular tissue to the eye of the observer. Many other possibilities of reflected or transmitted colors can also be envisioned for protecting the ocular tissue or underlying tissue, for beauty or aesthetic reasons or for enhancing light and/or other forms of energy penetrating the ocular tissue for therapeutic or diagnostics or research purposes.

### EXAMPLE 5

### Multi-Photon (MP) Method and Device for Enhanced Penetration

A series of cavities can be generated in the z-axis direction. These can be inter-connected to create an effective channel for the delivery of drugs or medicine or for example, for the storing of drugs, medicine, nutrients and vitamins within the tissue or ocular tissue for disabling muscles, for the control of diseases such as diabetes or other ocular tissue diseases.

Such porosity-generating or enhanced drainage patterns can be made from the bottom up. For example by three dimensional subsurface perpetrations of cavity or discontinuity in the tissue, a series of cavities or spaces or disruptions in tissue continuity can be made to allow beneficial substances such as drugs or liquids or vitamins to be inserted. The plurality of cavities and subs surface pores can be connected to the surface by a plurality of “wells” or channels or drilled holes that leads from the pores to the surface. A porous array of this type can allow a novel method for treating ocular diseases or using organs within the eye as a storage reservoir for medicine drugs, or nutrients among other beneficial substances.

### EXAMPLE 6

### Direct Modification to the Ocular Tissue which Can be Made by Creating the Following Pattern of Volumetric Micro-Zones

For the creation of three dimensional spatial patterns for the storage of medications or nutrients or other beneficial fluids, spot sizes of from about 5 μm to about 50 μm can be more desired.

Additionally and preferably, if for example a series of tissue modified spots are created, spacing between the edges of each spot of modified material thus created can vary in diameter, for example, from about 0.01 μm to about 5 mm, from about 0.1 μm to about 1 mm and more preferable from about 0.2 μm to about 500 μm and more preferably yet form about 0.2 μm to about 200 μm and more preferably from about 0.3 μm to about 100 μm, and more preferably yet from about 0.4 μm to about 50 μm, and more preferably yet from about 0.5 μm to about 25 μm, and more preferably from about 0.7 μm to about 10 μm, and more preferably from about 1 μm to about 5 μm.

In a preferred alternative embodiment, continuous lines can be created. A line pattern with individual line diameters ranging from about as little as 0.1 μm to about as much as 5 mm, from about 1 μm in diameter to about 100 μm in diameter and extending over the entire targeted area wherein a fractional surface area of from about 1% to about 99%, from about

Excising Tumors and Ocular Tissue Within the Eye.

In a preferred embodiment a method and a device for the creation of fresh surface with minimal or no thermal damage is also contemplated. As shown in FIG. 7,an energy source 200 capable of generating pulses of electromagnetic energy with wavelength between 100 micrometer and 100 nm and preferably between 2 micrometer and 200 nm and most preferably between 1.6 micrometer and 330 nanometer is sent directly to a beam modifier 196 or to an amplifier 210. Each pulse arriving at the beam modifier 196 has energy of between 0.01 nj and 100 Joule and preferably between 1 nj and 10 Joules and most preferably between 10 nJ and about 500 mJ.

The pulse coming out of the amplifier 210 may have a repetition rate of 0.01 Hz to 50 MHz and preferably between 1 Hz and about 1 MHz, and most preferably between about 1 Hz and about 100 KHz. The pulse train out of the oscillator 200 has a pulse repetition rate of between about 100 KHz and about 200 MHz and preferably between 10 MHz and 100 MHz.

The beam 133 or 215 are either stirred by a mirror-scanner 103 in the beam modifier 196 or by beam modifying elements such as diffractive optics a multiple lens arrays or Kinoform phase plates within the beam modifiers 196 to create a multiple beam arrays 223 of at least 9 beams, capable of creating a pattern of focused beam spots at the surface 143. The focused beam spots should have sufficient energy density to ablate at least some of the material from the surface of the target. Subsequent beam pulses remove additional material preferably in a way that leave little or no thermal or mechanical damage on the remaining material, preferably, such thermal or mechanical should be confined to material extending to less than about 50 micrometer from the ablated surface and more preferably to less than about 7 micrometer and most preferably to less than about 2 micrometer.

In further preferred embodiment the method contemplated herein also consist removing the surface layer of the targeted surface by a multiple beam as described above and shown in FIG. 7. The exposed surface is then photographed or otherwise imaged with a CCD camera or optical coherent tomography or fluoresce imaging, or nonlinear/third harmonic generation (THG) imaging, multiphoton imaging, Birefringent imaging, Fluorescence lifetime imaging, infrared thermography, or other method of imaging or other appropriate imaging system 134 as shown in FIG. 7. The imaging is designed to look at signs of cancer, malignant cells, excess pigmentation, vascular target, or other ocular tissue cells abnormality. For example, in a preferred embodiment, a baseline image or an image of adjacent normal ocular tissue surface can be obtained and stored in the system data base. The obtained image is then compared to the desired surface characteristics (for example, a surface image free of signature marker of malignant cells (for example, Florence signature of malignancy). An automated microprocessor 202, in a preferred embodiment, then determines if the desired image was obtained. If such an image has not achieved, another layer of the ocular tissue surface is removed, by the beams 223. Alternatively and preferably, the use of the system can control and inspect the image manually. For example, the image from the monitoring element 134 may appear on a screen 204. The operator 206 (for example, a physician) can then examine the image activate an on/off switch 207 to continue removing several layers of ocular tissue with the beams 223 or stop the treatment process. The process can be repeated iteratively until the operator decide that sufficient volume of tissue has been removed so that the image he receives no longer show the undesired parameter, for example, fluorescence from cancer cells within the surface of the eye. At that point the operator can decide to stop the process and does not remove additional layers of tissue. Alternatively and preferably, the ablation and removal of surface tissue layers can be controlled by the microprocessor 202 so the process is automated as described above.

The method and the device shown in FIG. 7, can thus be used to ablated and image a 3-dimensional target tissue, ablating the surface, imaging the exposed surface, removing additional layers. For example, a multi-photon laser scanning microscope can be used for the imaging process. As described above, in a preferred embodiment, the oscillator 200 generate ultra-short laser pulses shorter than about 10 ps and the amplifier 210 increase the energy of these pulses.

Thus, a preferred embodiment include ablating or removing ocular tissue surface with pulses form the amplifier 210 with pulse energy of about 100 nJ or more and more preferably with pulses of 1 microjoule or more, and subsequently use un-amplified pulses from the oscillator with pulse energy of about 1 microjoule or less and more preferably pulses of 100 nJ or less. If the images indicated that more tissue needs to be removed then the pulses from the amplifier with additional energy can be used. In a preferred embodiment, prior to imaging, the ocular tissue is stained to allow better imaging. In yet another preferred embodiment, the stain includes a fluorescent material applied to the exposed ocular tissue surface. In yet another preferred embodiment, fluorescent labeled antibodies are applied to the tissue ablated by the laser or an immuno-reactive antibody can be applied to the ablated by the laser. In further preferred embodiment the amplifier 210, is an optical amplifier, said optical amplifier can be, for example, a regenerative optical amplifier, multi-pass optical amplifier, fiber amplifier, a semiconductor optical amplifier, optical parametric amplifier (OPA), or other type of amplifier.

In yet another preferred embodiment, the ablation or surface layers removal step consist of removing at least 2 micrometer of the ocular tissue surface tissue. More preferably, at least about 5 micrometers of the ocular tissue surface is removed during each removal step. More preferably yet, at least 10 micrometer of ocular tissue surface tissue is removed. More preferably yet a layer of at least 15 micrometer is removed. In a most preferred embodiment a layer of at least 20 micrometer is removed.

In a further preferred embodiment, the device contemplated is used for biopsy and imaging of three dimensional section of the targeted ocular tissue, the device comprises as shown in FIG. 7, a source of energy 200 capable of producing a short light pulses, preferably shorter than about 1 ns, said device further comprises a multiple beams pattern of at least three beams, 223, the device further comprises, means to generate an image of the ocular tissue surface, before during and after ablating the surface with said energy source 200 and beam 223. Preferably the device further comprises an imaging element 134, for example, a CCD camera, a photomultiplier, an Optical Coherent Tomography, a microscope, and ultrasound detector, a photo-acoustic imaging element, or other elements capable of imaging the ablated and exposed surface, said imaging element 134, is capable of sending the detected signals to a microprocessor 202 operatively coupled to the energy generator (for example, an ultrashort pulse laser) 200, to automatically process the image and decide if sufficient depth or sufficient amount of ocular tissue was removed. Preferably, the device can also comprise a monitor 204 which shows the image and data collected by the detector or imaging element 134, wherein an operator 206 can view the data and image from the freshly exposed ocular tissue and decide if another layer of ocular tissue has to be exposed or if the operation can be terminated. The device may further comprise a computer microprocessor 202 that can automatic repeat the ocular tissue layer removal process and the process of generating images of subsequently exposed ocular tissue surfaces until a desired volume of the ocular tissue 143 has been removed (for example, all the malignant or benign but abnormal ocular tissue) or until a sufficient amount of tissue has been imaged.

In a preferred embodiment, the device further comprises an acquisition and storage component 202, for example a computer with acquisition, processing and storage capacity. The device may further comprise the ocular tissue endogenous signals for imaging.

In a preferred embodiment the device further utilize optical nonlinearities such harmonic generation, (second or third harmonic generation), Raman spectroscopy, multi-photon absorption fluorescence and multiphoton spectroscopy, or nonofficial microscopy to generate image contrast or image of the exposed surface. In further preferred embodiment, said multiple images are combined to generate three dimensional image of the volume of the ocular tissue. In further preferred embodiment, said images are viewed consecutively by the operator who then decides whether sufficient amount of tissue was removed or whether all the malignant tissue was removed. Preferably, in another preferred embodiment, the operator relies on automated processor 202 to decide where a targeted tissue volume or a segment of a tissue with specific physical characteristics that can be distinguished by the detector 134 and the processor, 202, has been removed so that the operation of the device can be stopped.

Ocular Tissue Tumor Removal

In a preferred embodiment the iterative removal and imaging process described above is replaced with iterative ocular tissue removal and diagnostics and analysis of tissue in procedures or surgeries to remove benign or cancerous tumors.

for ocular tissue cancer removal in anatomic areas where maximum preservation of healthy tissue is desired for safety and functional purposes (the retina, the eyelids, or other regions of the eye tissue), for cancers with indistinct margins, and for recurrent caencrs in scar tissue. It is especially indicated for lesions that have recurred following prior treatment, or for lesions in anatomic areas that have the greatest likelihood of recurrence.

In a preferred embodiment, the device of FIG. 7 is used to remove ocular tissue tumor (malignant or benign) for example, such as those done in ocular surgery, and is preferably performed with the following steps: Surgical removal of tissue (Surgical Oncology) with the device of FIG. 7 2A; Mapping the targeted region of tissue, ablating or photo-modifying malignant tissue, analyzing the emission, OCT, microscopic, Breakdown emission, fluorescence emission, SHG, THG, or other information carrying signal detected by the device sensors, repeating said modifying, ablating or photo-disruption step on the targeted tissue, (for a non-limiting example, modifying the tissue between 5 and 10 micrometers using a EME pulse),

The device of FIG. 7 used to surgically remove ocular tissue from the suspected ocular tissue region. After each surgical removal or modification of tissue, the signals from the sensors monitoring the specimen are analyzed by the computer program processor and the removal or modification steps using EME pulses is repeated as needed as long as tissue containing signals characteristics of a tumor (for example, Spectroscopic breakdown signal, color, or fluorescence imaging, etc.). If cancer or tumor markers are found, its location is identified by the imaging and sensors module, and the surgeon continue with the treatment. This procedure is repeated until no further signals or characteristics of tumor or cancer is found.

Additional preferred embodiment utilize the device for creating the disruption below the surface described above to generate as many physical disruption in the path of an external influence many ocular tissue targets. For example, in a preferred embodiment such pattern of photodisruption is generated below the surface of the cornea, sclera or crystalline lens (hereunder—CL) tissue and down to a depth of 100 um. If we assume disruptions diameter of 1 to 3 micrometer. For example, we consider disruptions of a diameter of about 1.5 micrometer in diameter and a 1.5 micrometer of unmodified tissue between said photodisruption for a total of 3 micrometer. This means that about 33 layers of photo-disruption spots can be created down to a depth of about 100 micrometer. If for simplicity we assume 20 such layers of photo disruptions generate in the targeted tissue 181 shown in FIG. 7, then in a 1 cm2 area we can, for example, generate two “disruption cell” in 10 um (e.g. disrupted+untouched for example of 5 micrometer “disruption cell” size). This means that 2000 disruptions can be generated in a linear pattern one cm long and in an area of 1 cm2 we have 4 million disruptions, and in a volume of 100 um by 1 cm square, we have about 80 million such disruptions. Since an optical oscillator that can generate a picosecond or femtosecond light pulses, often operates at pulse repetition rate of from about 70 MHz to 90 MHz, for example, 88 MHz, such 80 million subsurface disruption can be generated in less than one second. If for example a surface of the ocular tissue of area of 100 cm2 needs to be covered, for example a surface of the facial area, than such ultrashort pulse laser, or a picosecond laser, or a microchip laser, can generate such a photo-disruptions pattern in about 100 seconds or about a minute and half which is a reasonable operating time for a treatment.

In another preferred embodiment shown in FIG. 7, a device and a method for creating the disruption below the surface is described. The method include the step of using the device described in matmat FIG. 7 to create a photo-disruption at least five micrometer below the surface of the ocular tissue and at least five micrometer above the lower boundary of the ocular tissue target. In a preferred embodiment, such disruptions leave at least the upper five micrometer of tissue from the upper surface of the ocular tissue unchanged and at least five micrometer of above the lower boundary of the tissue.

Another preferred embodiment contemplates creating a zone of photo-disruption below the surface of the ocular tissue while leaving layer at least 10 micrometer below the surface physically unchanged and at least 10 micrometer above the lower boundary of the tissue.

Another preferred embodiment contemplate the device and method shown in FIG. 7 and discussed above, for creating the zone of photodisruption spots below the ocular tissue surface as described above wherein a physical characteristic selected from the group of: Thermal conductivity, Heat capacity, Porosity, Electrical conductivity, Elasticity, The ability to allow fluid to flow across the ocular tissue from the surface of the eye to deeper ocular tissue layers or ocular tissue components; The ability to allow liquid to flow across the ocular tissue from the surface to deeper regions of the tissue; The ability to allow a substance to flow across the ocular tissue from the surface to the other ocular organs or tissue components; Other physical properties of the ocular tissue is changed according to the same change parameters as described above for.

Another preferred embodiment created the above changes in deeper layers of the tissue, for example, said zone of photo-disruptions is created within the cornea or the crystalline lens.

Eye Surgery Device and Method

A device for use in laser surgery includes a laser system, a disposable contact window and means to steer the laser beam to create a pattern of surface or subsurface tissue alteration to enhance the properties of the tissue.

A series of photodisruption, ablation, voids, or photocoagulation spots are created.

The spots can be as small as 0.01 micrometer to as much as 3 mm and preferably from about 0.05 micrometer to about 70 micrometer.

These spots can be generated at or below the surface of the ocular components or components of the eye tissue. In a preferred embodiment, said spots can be made from the surface to a depth of about 20 mm and preferably from about 5 micrometer below the surface to about 4 mm below the surface and more preferably from about 10 micrometer below the surface to about 500 micrometer below the surface of the components of the eye, and most preferably from about 15 micrometer below the surface to about the location of the posterior surface of the organ being modified.

An adjustable contact ring for enhancing the contact of the window with the laser system may also be employed. The contact window is aligned with the targeted tissue.

The disposable window ensures the creation of a surface or a subsurface pattern in target material, using laser or energy source surgery, the device includes optical elements which creates a controlled air to window interface that helps assure an accurate localization of the focal spot inside the tissue.

An Index Matching/Pattern Positioning Window

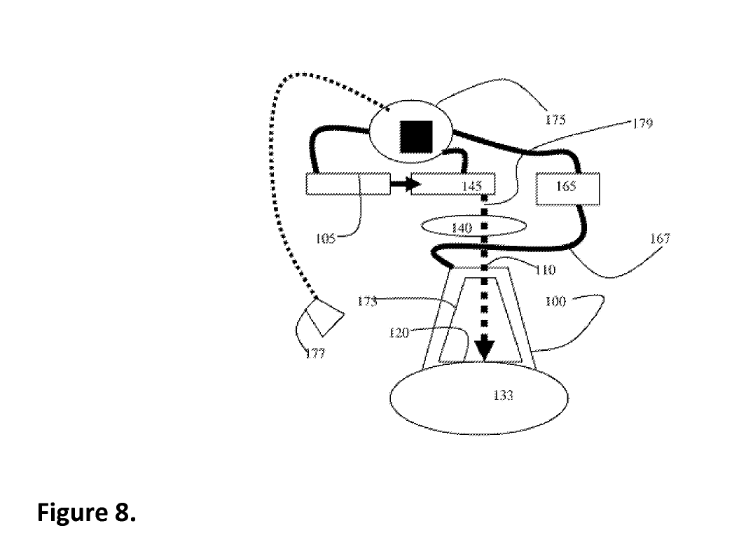

FIG. 8 shows a preferred embodiment of a device 100 for use in laser or energy-based surgery comprises: An energy source or a laser 105, a window member 100 having a substantially flat first surface 110 facing the incoming beam, and a substantially deformable second surface 120, said second surface being able to conform to the surface of the target material 130 when pressed against the target material.

The system of above may also comprise said window and lens system consisting of an optical index of refraction that match the index of refraction of target material.

The lens and window system may thus allow an incoming laser beam to be focused on or under the surface of the target material with minimal scattering.

The system above may also comprise a sensor member 135 allowing the user to determine the distance form a focusing member 140 and the first surface of the said lens and window system 100.

The sensor member may comprise meter stick, an optical gauge, an OCT, an optical radar, a radar, or other member capable to measure a distance.

The system above may also comprise a mirror or a scanner 145 capable of moving the position of the beam 150 along said first surface 110.

The system of claim 1 wherein said window and lens system is attached to the compartment of the energy source or laser 105.

The System may further comprise said window and lens system that are filled with fluid.

The system described herein may further comprise the beam from the energy source is adjusted to focus at a predetermined depth below said first surface on or below the surface of the target.

The system may further comprise of said second surface 120 that is able to communicate at least some fluid from said window and lens interior to said target surface.

The system may further comprise of said second surface 120 which is also able to communicate suction to said target surface 133.

The system may further comprise a pump 165 said pump is able to generate said suction provided at the second surface 120 to the target surface 133. For example, a member such as s tube or a hollow guide 167 can conduct suction or removal of air or positive pressure or air flow to or from the desired target.

The system may further comprise a member made of medical grade material, for example a window or a lens member 100. The window or lens member 100 is place in contact with the target material for example the surface of the eye, or cornea, 133. The contact Window or lens member 100 can additionally have an inner chamber 173 where a suction or negative pressure is created and applied to the target material, for example the surface of a cornea 133. A controller 175 receives feedback signal from sensors 177, for example an Optical Coherent tomography, a camera, a luminescence or fluoresce detectors, a confocal microscope, a microscope or magnifiers, thermal imager or thermocouple, IR detector, IR camera, transducers or other useful sensing devices. The controller 175 also controls the energy sources 105, 145, the suction or pressure applying elements 165 the position of the window 100, the beam modifier 140 and the parameters of the beam 179

Another embodiment contemplates a device for creating predetermined pattern of spots of modified material properties on or below the surface of a target comprising an energy source, a member capable of focusing the beam of energy at the desired location, a member capable of moving the energy beam in a predetermined pattern.

Another embodiment contemplates a device for creating predetermined pattern of spots of modified material properties on or below the surface of a target comprising an energy source, a member capable of focusing the beam of energy at the desired location, a member capable of moving the energy beam in a predetermined pattern.

According to the present embodiment, the device above may remove the following depth of material with each interacting pulse (all depths are in micrometers):

0.5 um, 1 um, 2, 3, 4, 5, 7, 10, 12, 15, 17, 20, 25, 30, 35, 40, 45, 50, 60, 70, 80 90 100 110 120 130 140 150 170 185 200 220 230 250 260 275 285 300 325 350 375 400 450 500 550 600 650 700 750 800 850 900 950 1000 um,

Further, the device above may also remove the following greater depth of material with each interacting pulse (all depth are in millimeters): 1 mm 1.2 mm 1.5 mm 1.7 mm 2 mm 2.5 mm 2.75 mm 3 mm

The total amount of material removed with each pulse is the above depth times the area corresponding to the ablated spot size.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A device for treating tissue or the human eye, the device comprising: an energy source coupled to at least one optical component and at least one controller that operates the energy source, to generate an output beam of energy, said beam of energy generates a plurality of photomodified volumes of material at or below the surface of the eye, wherein said photomodified volumes comprise a three dimensional pattern within the tissue treated tissue or human eye, and wherein said three dimensional pattern thus generated further shows no unintended modifications in the targeted tissue of the eye further than about 5 micrometer outside the boundary of said pattern of photomodified volumes.

2. The device of claim 1, wherein the controller is configured to control the output beam such that the output energy beam comprises pulsed emission, wherein each pulse has a duration of no more than about 10.sup.−6 seconds.

3. The device of claim 1, wherein the controller is configured to control the output beam such that the output energy beam comprises pulsed emission, wherein each pulse has a duration of no more than about 10.sup.−9 seconds.

4. The device of claim 1, wherein the controller is configured to control the output beam such that the output energy beam comprises pulsed emission, wherein each pulse has a duration of no more than about 50×10.sup.−12 seconds.

5. The device of claim 1, wherein the optical components comprise at least one diffractive optic component capable of producing at least 10.sup.3 interaction spots in the x, y plane of the targeted tissue.

6. The device of claim 1, wherein the controller produces the plurality of cavities that include an overlapping photomodifications or photomodifications wherein the spacing between the edges of the the photomodified tissue is from about 0.01 micrometer to about 5 mm.

7. The device of claim 1 wherein said unintended modification comprises one or more unintended modification from a group including: Thermal modifications, Mechanical modifications, Chemical modifications, Structural modifications, and any other modification to the structure composition or characteristic of the native, unmodified tissue.

8. The device of claim 1 wherein said photo-modifications or photodisruptions comprises at least one or more from a group including: Thermal modifications, Mechanical modifications, Chemical modifications, Structural modifications, and any other modification to the structure composition or characteristic of the native, unmodified tissue.

9. A device for treating a region of a targeted region of the eye, the device comprising: an energy source coupled with at least one optical component capable of directing sources energy into multiple locations in the targeted region; and a controller that operates the energy source to direct said plurality of photomodified spots below the surface of the target and create a plurality of photomodified spots below the surface in a 3-dimensional pattern within the thickness of the target without any thermal damage further than 5 um below the boundary of the plurality of photomodified spots.

10. The device of claim 9, wherein the controller is configured to configure the source output energy such that each beam has a pulse duration of no more than 10.sup.−9 seconds.

11. The device of claim 9, wherein the source energy produces at least 1000 photomodified spots in in a plane perpendicular to the optical axis of the eye.

12. The device of claim 9, wherein the source energy produces at least 1000 photomodified spots in a plane parallel to the optical axis of the eye.

13. The device of claim 9 wherein the photomodified spots change the mechanical characteristics of the eye.

14. The device of claim 9 wherein the photomodified spots change the optical characteristics of the eye.

15. A method of modifying a region of eye, comprising: providing a laser beam generator, directing the beam from the beam generator to produce a plurality of laser-tissue interaction spots at or below the surface of the eye; wherein the laser-tissue interaction spots produce no thermal damage farther than 5 .mu.m from the modified tissue, whereby the plurality of laser-tissue interaction spots create scattering centers to decrease the amount of subsequent light energy penetrating the eye.

16. The method of claim 15, wherein the plurality of laser-tissue interaction spots comprise a density of at least Ten laser-tissue interaction spots per cubic millimeter in the region of the tissue of the eye.

17. The method of claim 15, wherein the plurality of laser-tissue interaction spots have a diameter ranging from about 0.1 micrometer to about 20 micrometer.

18. The method of claim 15, wherein the plurality of laser-tissue interaction spots are at least 90% non-ablative.

19. The method of claim 15, wherein the plurality of laser-tissue interaction spots comprise spacing between the edge of adjacent laser-tissue interaction spots ranging between about 0.01 micrometer to about 10 mm.

20. The method of claim 15, wherein the plurality of laser-tissue interaction spots comprise at least some overlap between adjacent laser-tissue interaction spots.
